(19) United States
(12) Patent Application Publication
(10) Pub. No.: US 2024/0123004 A1
(43) Pub. Date: Apr. 18, 2024

(54) ONCOLYTIC VIRUS FOR SYSTEMIC DELIVERY AND ENHANCED ANTI-TUMOR ACTIVITIES

(71) Applicant: UNIVERSITY OF HOUSTON SYSTEM, Houston, TX (US)

(72) Inventors: Shaun ZHANG, Houston, TX (US); Xinping FU, Houston, TX (US)

(21) Appl. No.: 18/264,737

(22) PCT Filed: Feb. 8, 2022
(86) PCT No.: PCT/US2022/015703

§ 371 Date: Aug. 8, 2023

Related U.S. Application Data
(60) Provisional application No. 63/263,528, filed on Nov. 4, 2021.
(60) Provisional application No. 63/200,011, filed on Feb. 9, 2021.

(51) Int. Cl. A61K 35/763; A61P 35/00; C07K 14/705; C12N 7/00

(52) U.S. Cl. CPC A61K 35/763; A61P 35/00; C07K 14/70503; C12N 7/00; C12N 2710/16632; C12N 2710/16651

(57) ABSTRACT

This invention relates to oncolytic viruses which are more resistant to neutralization and phagocytosis by immune systems, and methods for their preparation and treatment of disorders and diseases (such as cancer) with them. Described herein is an oncolytic Herpes Simplex Virus Type 1 (HSV-1) or Herpes Simplex Virus Type 2 (HSV-2) treated in immune sera that contain a high level of anti-HSV antibodies. In one preferred embodiment, the oncolytic virus includes an extracellular CD47 domain inserted into the N-terminus of a glycoprotein in order to inhibit phagocyte activity. The oncolytic virus is suitable for systemic administration for the treatment of cancer.

This application is the U.S. national stage of International Patent Application No. PCT/US2022/015703, filed Feb. 8, 2022, which claims the benefit of U.S. Provisional Application No. 63/200,011, filed Feb. 9, 2021, and U.S. Provisional Application No. 63/263,528, filed Nov. 4, 2021, each of which is hereby incorporated by reference.

### STATEMENT OF FEDERALLY SPONSORED RESEARCH OR DEVELOPMENT

This invention was made with government support under grant no. G110207 awarded by the National Institutes of Health. The government has certain rights in the invention.

### SEQUENCE LISTING

The instant application contains a Sequence Listing which has been submitted electronically. The contents of the electronic sequence listing (sequencelisting.txt; Size: 43,713 bytes; and Date of Creation: Feb. 8, 2022) is herein incorporated by reference in its entirety.

### FIELD OF THE INVENTION

This invention relates to oncolytic viruses which are more resistant to clearance and neutralization by immune systems, and methods for their preparation and treatment of disorders and diseases (such as cancer, including metastatic cancer) with them.

### BACKGROUND OF THE INVENTION

Cancer virotherapy is based on a pragmatic approach with a simple and practical therapeutic mechanism—exploiting the intrinsic infection/cytolytic activity of a virus for the selective destruction of malignant cells. Significant progress has been made in recent years on developing cancer virotherapy. Currently, one oncolytic virus derived from type I herpes simplex virus (HSV-1), Imlygic or T-VEC, has been approved for clinical application for the treatment of unresectable cutaneous, subcutaneous and nodal lesions in patients with melanoma recurrent after initial surgery (Greig, 2016). Additionally, there are multiple ongoing clinical trials testing the efficacy of a variety of oncolytic viruses constructed from different viruses (Macedo et al., 2020).

Currently, oncolytic virotherapy is predominately administered by the intratumoral route. However, systemic delivery is desirable for treating patients with many malignancies, particularly for those who have developed metastatic diseases. In fact, the overwhelming majority of patients who receive virotherapy will likely have metastatic disease. Despite the importance of this delivery route, an optimal systemic delivery strategy has not yet been developed. Studies have shown that, although the therapeutic effect was detected in some experiments after oncolytic HSVs were delivered by the systemic route, the overall therapeutic efficacy was significantly less than those seen with intratumoral delivery (Fu et al., 2006; Nakamori et al., 2004). Several key components are impeding the delivery efficiency of oncolytic viruses by the systemic route.

First, the anti-HSV neutralizing antibodies, which can be either pre-existing due to the patient's previous exposure to the virus or freshly developed from repeated administration of the oncolytic virus during treatment, can bind to the introduced viral particles. This prevents the viruses from infecting tumor cells as well as resulting in their rapid clearance by the host's antibody-dependent cellular phagocytosis (ADCP) (Huber et al., 2001; Tay, Wiehe, and Pollara, 2019). Indeed, animal studies have shown that the pre-existing humoral immunity is detrimental to the infectivity of oncolytic HSVs (Fu and Zhang, 2001).

The anti-HSV neutralizing antibodies are found to be mainly targeted at two of the virus-encoded glycoproteins—glycoprotein D (gD) and gB (Cairns et al., 2015; Cairns et al., 2014). Both gD and gB contain many neutralizing epitopes, presenting at diverse orders of immunodominant hierarchy in different individuals (Bender et al., 2007). Overall, these epitopes are well-conserved among infected individuals and/or different species (Eing, Kuhn, and Braun, 1989). The studies have also indicated that HSV-2 is more difficult to neutralize than HSV-1 (Silke Heilingloh et al., 2020). The presence of strong epitopes presents as a major obstacle for systemic administration of HSV-based oncolytic viruses as neutralizing antibodies can readily recognize them and abolish the therapeutic activity.

Another important limiting factor that reduces the effectiveness of systemically delivered oncolytic virotherapy is the host's mononuclear phagocyte system (MPS). It has been shown that, after systemic delivery, the viral particles could be quickly cleared by MPS (Ellermann-Eriksen, 2005; Hume, 2006; Van Strijp et al., 1989). Specifically, studies by Fulci et al. have shown that depletion of macrophages can profoundly improve the therapeutic effect of oncolytic HSV (Fulci et al., 2007).

Yet another significant antiviral mechanism that can reduce the effectiveness of viral delivery with HSV is natural killer (NK) cells (Alvarez-Breckenridge et al., 2012a; Alvarez-Breckenridge et al., 2012b). The viral glycoprotein E (gE) is known to bind to IgG (Dubin et al., 1994). Recent studies have shown that NK cells can recognize HSV or HSV infected cells via the the binding of the surface CD16 activating receptor with the Fc region in IgG bound to gE (Dai and Caligiuri, 2018).

One way to limit the effect of neutralizing antibodies on the infectivity of oncolytic viruses is by mutagenizing each of the major epitopes. Indeed, a recent study has shown that mutagenizing two of these epitopes from the gD of an oncolytic HSV can indeed abolish the neutralization by the corresponding monoclonal antibodies (mAbs) (Tuzmen et al., 2020). However, both gB and gD of HSV-1 and HSV-2 are the major targets of the neutralizing antibodies and each of them contain almost a dozen of neutralizing epitopes. Using a traditional way of mutagenizing each of these epitopes from two glycoproteins in the context of a viral genome of over 150 kb long is not really practical. Moreover, this approach only deals with one of three major obstacles facing systemic delivery of oncolytic virotherapy—the neutralizing antibodies, and leaves the MPS untouched.

U.S. Pat. Pub. No. 2012/0301506 discloses the construction of FusOn-H2, an HSV-2 derived oncolytic virus, and its usage in treating malignant tumors. Administration of FusOn-H2 induces the patient's innate immune responses to tumor cells via neutrophils, which are able to destroy tumors efficiently when they migrate to the tumor mass. With the induced innate antitumor immunity, FusOn-H2 is effective at eradicating tumors even when it is used at very low doses.

U.S. Pat. Nos. 10,039,796 and 8,986,672 discloses a composition and use of a modified Herpes Simplex Virus Type 2 (HSV-2) for the treatment of cancer. The modified HSV-2 comprises a modified/mutated ICP10 polynucleotide encoding a polypeptide having ribonucleotide reductase activity and lacking protein kinase activity.

Fu et al., Oncotarget, 2018, 9(77):34543-34553, describes genetically engrafting CD47 to the membrane envelop of an oncolytic HSV to enable it to escape from the MPS.

There remains a need for oncolytic viruses suitable for systemic administration.

### SUMMARY OF THE INVENTION

The present invention is directed to oncolytic viruses suitable for systemic administration which are resistant to the immune clearance of a subject. Described herein are methods for a series of modifications introduced to a herpes simplex virus, such as herpes simplex virus-1 (HSV-1) or herpes simplex virus-2 (HSV-2) based oncolytic virus (such as FusOn-H2), which enable virus to escape from the clearance and neutralizations which occur in a subject. These modifications include: 1) a series of passage of FusOn-H2 in an immune serum or sera that contain high level of anti-HSV antibodies, and optionally 2) inserting the extracellular domain of CD47, a molecule that contains a “don't eat me” signal, into the N-terminus of the glycoprotein C (gC). Consequently, the modified virus, FusOn-SD, is significantly more effective than the parental FusOn-H2 at infecting and lysing tumor cells when administered by the systemic route.

Detailed analyses on FusOn-SD have revealed several novel findings. A prominent finding is the near complete absence of gE from the viral particles. This allows the virus to escape from the NK cell-mediated antiviral mechanism. The absence of gE from viral particles was only found in CD47-containing virus (e.g., FusOn-CD47) after serial passages in the presence of antiviral sera, rather than in a non-CD47-containing virus (e.g., FusOn-gC-Luc) that was subjected to a similar passage in the presence of antiviral sera. Another novel finding is that the insertion of the extracellular domain of CD47 into the N-terminus of gC was also found to enable the virus to escape neutralization by anti-HSV antibodies, even without passaging. These modifications and the unexpected consequential changes have significantly increased the capability of the oncolytic virus in treating cancer by systemic delivery without compromising its replication and safety.

One embodiment is a composition comprising an oncolytic Herpes Simplex Virus Type 1 (HSV-1) or Herpes Simplex Virus Type 2 (HSV-2), wherein the oncolytic HSV1 or HSV-2 is prepared by passaging at least twice an oncolytic HSV1 or HSV-2 with immune sera having elevated levels of anti-HSV antibodies. In one preferred embodiment, the oncolytic HSV1 or HSV-2 has a membrane envelope comprising glycoproteins, wherein at least one glycoprotein comprises an extracellular CD47 domain (such as the extracellular CD47 domain comprising amino acids 19-141 of CD47 (SEQ ID NO: 21)) inserted into the N-terminus of the glycoprotein. The glycoprotein may be selected from glycoprotein C, glycoprotein B, glycoprotein D, glycoprotein H, glycoprotein G, glycoprotein L, or any other viral membrane protein. For instance, in one embodiment, the glycoprotein is glycoprotein C. Without being bound by any particular theory, the inventors believe that the passaging in the immune sera mutates neutralizing epitopes on glycoprotein B and glycoprotein D or other viral genes of the oncolytic HSV-1 or HSV-2, or force other changes of these membrane proteins.

In another embodiment, the immune sera is a mixture of rat sera and human sera that has elevated levels of anti-HSV antibodies.

The composition comprising the oncolytic HSV-2 may be prepared by passaging seven times an oncolytic HSV-2 in the presence of rat sera having an elevated level of anti-HSV antibodies, followed by passaging seventeen times in the presence of a mixture of rat sera and at least one human serum having an elevated level of anti-HSV antibodies. The oncolytic HSV-2 can be prepared by passaging seven times an oncolytic HSV-2 in the presence of rat sera having an elevated level of anti-HSV antibodies followed by passaging twenty-three times in the presence of a mixture of rat sera and at least one human serum having an elevated level of anti-HSV antibodies.

In one embodiment, the oncolytic HSV-1 or oncolytic HSV-2 comprises a modified ICP10 coding region lacking nucleotides 1 to 1204 of an endogenous ICP10 coding region, wherein the oncolytic HSV-1 or HSV-2 comprises the modified ICP10 operably linked to an endogenous or a constitutive promoter and expresses a modified ICP10 polypeptide that lacks protein kinase (PK) activity but retains ribonucleotide reductase activity. Preferably, the oncolytic HSV-1 or HSV-2 is capable of selectively killing cancer cells.

In one embodiment of the compositions described herein, the composition comprises an oncolytic HSV-2 which was prepared by passaging FusOn-H2 oncolytic virus.

In another embodiment, the oncolytic HSV1 or HSV-2 having an extracellular CD47 domain is free or substantially free of gE.

Another embodiment is a method of preparing a composition comprising an oncolytic Herpes Simplex Virus Type 1 (HSV-1) or Herpes Simplex Virus Type 2 (HSV-2) comprising passaging at least twice an oncolytic Herpes Simplex Virus Type 1 (HSV-1) or Herpes Simplex Virus Type 2 (HSV-2) with immune sera that has elevated levels of anti-HSV antibodies. The oncolytic HSV-1 or HSV-2 which is to be passaged as well as the immune sera may be as described herein. Serum with elevated levels of anti-HSV antibodies can be obtained from animals vaccinated for HSV (e.g., HSV-2). In one embodiment, the method comprises passaging the oncolytic HSV-1 or HSV-2 at least two times in the presence of rat sera followed by passaging at least once in the presence of a mixture of rat sera and at least one human serum that has elevated levels of anti-HSV antibodies. In another embodiment, the method comprises passaging seven times the oncolytic HSV-1 or HSV-2 in the presence of rat sera having elevated levels of anti-HSV antibodies followed by passaging seventeen times in the presence of a mixture of rat sera and at least one human serum having elevated levels of anti-HSV antibodies. In yet another embodiment, the method comprises passaging seven times the oncolytic HSV-2 in the presence of rat sera having elevated levels of anti-HSV antibodies followed by passaging twenty-three times in the presence of a mixture of rat sera and at least one human serum having elevated levels of anti-HSV antibodies.

In a preferred embodiment of any of the compositions or methods described herein, the oncolytic HSV-2 to be passaged is FusOn-H2 oncolytic virus.

In yet another preferred embodiment of the compositions or methods described herein, the oncolytic HSV-2 to be passaged is FusOn-CD47 oncolytic virus.

Yet another embodiment is a method of treating cancer (for instance, metastatic cancer) in a patient in need thereof with an HSV-based oncolytic virotherapy comprising administering a composition or HSV-1 or HSV-2 oncolytic virus described herein. In one embodiment, an effective amount of the composition is administered. In a preferred embodiment, the composition is systemically administered. In certain embodiments, the composition may be used to treat a solid tumor. In one embodiment, the patient is vaccinated against HSV-1 and/or HSV-2. In another embodiment, the patient has HSV-1 and/or HSV-2.

### BRIEF DESCRIPTION OF THE DRAWINGS

For a more complete understanding of the present invention, including features and advantages, reference is now made to the detailed description of the invention along with the accompanying figures:

FIG. 1 is a bar chart showing differential sensitivity of trained and untrained FusOn-H2 to anti-HSV sera. 1×10⁴ untrained Fuson-H2 or trained FusOn-SS9 were mixed with either human or rat anti-HSV-2 sera at a final concentration of 1:5. After 1 h incubation at 37° C., the solution was applied to Vero monolayer at a 12-well plate. The plaques were countered 48 h later after crystal violet staining.

FIG. 2A is a schematic illustration of FusOn-cd47 construction strategy. It shows the insertion of EGFP-Luc gene cassette and the CMVp-HAtag-CD47ECD into the backbone of FusOn-H2 was done through homologous recombination, with the left flanking (LF) sequence and the 3′ region of gC (from the transmembrane domain to the polyA) as the right flanking sequence. The details of individual components in the gene cassette are depicted in the drawings and are labelled accordingly. The abbreviations are: CMVp, cytomegalovirus immediate early promoter; LTR, Rous Sarcoma long terminal repeats that contain the promoter region of this virus; GFP-Luc, EGFP-luciferase fusion gene; HA, HA tag; CD47, murine CD47 extracellular domain; gC, the complete gC coding region. LF, the left flanking region of gC. The recombinant viruses were identified by GFP expression and purified to homogeneity.

FIG. 2B is a schematic illustration of FusOn-cd47 construction strategy. It shows FusOn-Luc, which was used as a control virus, was constructed in a similar way, except that it does not contain the CD47 extracellular domain.

FIG. 3 is a chart showing the comparison of FusOn-CD47 and FusOn-Luc for ability to evade clearance by phagocytes. It is a Comparison of virus yield with or without the presence of phagocytes. Vero cells in 12-well plates were infected with either virus at 0.1 pfu/cell without or with the presence of 200,000 splenocytes. Cells were harvested 48 h and the virus yield was determined by plaque assay. *p<0.05 as compared with other three wells.

FIG. 4A is a chart showing the mean photon reading from daily IVIS imaging measurement, demonstrating that FusOn-CD47 can be more efficiently delivered than FusOn-Luc by the systemic route in a CT26 tumor model. The tumor was established at the right flank of Balb/c mice by subcutaneous implantation of CT26 cells. Once tumor reached the approximate size of 8 mm in diameter, 2×10⁶ pfu of either FusOn-CD47 or FusOn-Luc was given systemically.

FIG. 4B is a time-lapse of images of typical mice days after systemic delivery of FusOn-CD47. It shows that FusOn-CD47 can be more efficiently delivered than FusOn-Luc by the systemic route in CT26 tumor model. Tumor was established at the right flank of Balb/c mice by subcutaneous implantation of CT26 cells. Once tumor reached the approximate size of 8 mm in diameter, 2×10⁶ pfu of either FusOn-CD47 or FusOn-Luc was given systemically. Animals were imaged daily starting on day 2 for luciferase expression.

FIG. 5 is a chart showing enhanced resistance of FusOn-CD47 to the neutralization effect by the anti-HSV sera after training. 1×10⁴ untrained FusOn-CD47 or the trained FusOn-CD47-SS24 (7 passages in the presence of rat sera and 17 passages in the presence of rat sera plus one human serum) were mixed with either human or rat anti-HSV-2 sera at a final concentration of 1:5. After 1 h incubation at 37° C., the solution was applied to Vero monolayer at a 12-well plate. The plaques were countered 48 h later after crystal violet staining.

FIG. 6 is a chart showing trained FusOn-CD47 acquired additional resistance to anti-HSV immune sera. 5×10³ untrained Fuson-CD47 or the same virus trained at different stage [serial selection 24 (FusOn-CD47-SS24), serial selection 49 (HR49, N2-N5 and N7-N8)] were mixed with an eight-human sera mixture at different dilutions. The trained FusOn-H2 (FusOn-SS9) was also included in this experiment. After 1 h incubation at 37° C., the solution was applied to Vero monolayer at a 12-well plate. The plaques were countered 48 h later after crystal violet staining.

FIG. 7 is a bar chart showing that the insertion of CD47 can enhance the ability of an oncolytic HSV to escape neutralizing HSV antibodies. Five hundred pfu of FusOn-CD47 and FusOn-Luc were incubated with anti-HSV-2 sera at the indicated dilution (1:40 or 1:160) at 37° C. for 1 hour before they were used to infect Vero cells in a 6-well plate. The same number of viruses was incubated with medium only as a control. The cells were stained with crystal violet 48 hours later for enumeration of viral plaques. The percentage of plaques was calculated by dividing the plaque number in the wells containing anti-HSV sera with those from the wells with the same virus without the anti-HSV sera (the controls). ★ indicates a p<0.05 as compared with FusOn-Luc.

FIG. 8 is a time-lapse of images of typical mice days after systemic delivery of FusOn-CD47, FusOn-Luc or FusOn-SD. It shows that FusOn-CD47, but not FusOn-Luc, could be sufficiently delivered by the systemic route, to tumor-bearing mice that have pre-existing anti-HSV immunity, and the passaging of FusOn-CD47 in sera containing anti-HSV antibodies (to generate FusOn-SD) further enhanced the ability of the virus to be delivered by systemically in these mice. Balb/c mice were vaccinated with FusOn-H2 and then implanted with CT26 tumor cells at the right flank. Mice in separate groups were given with each of these three viruses via the tail vein at the dose of 1×10⁷ pfu. Mice were imaged using an IVIS imager at the indicated days after virus injection.

FIG. 9A is is a bar chart showing the absorbance at 450 nm of FusOn-H2, FusOn-SD, and PBS (negative control) after treatment with mouse anti-HSV-2 gE (1:1000 dilution) and HPR-conjugated rabbit anti-mouse IgG (1:10,000 dilution).

FIG. 9B is a western blot showing detection of gE in samples of FusOn-SD and FusOn-H2 after treatment with mouse anti-HSV-2 gE (1:1000 dilution) and HPR-conjugated rabbit anti-mouse IgG (1:10,000 dilution).

FIG. 9C is a schematic illustration on the mechanism of NK cell recognition of HSV or HSV infected cells via gE.

### DETAILED DESCRIPTION OF THE INVENTION

In describing preferred embodiments of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the figures.

### Definitions

The term “Herpes Simplex Virus” or “HSV” as used herein refers to an enveloped, icosahedral, double-stranded DNA virus that infects mammals, including humans. Wild-type HSV infects and replicates in both terminally differentiated non-dividing cells and dividing cells. “HSV-2” refers to a member of the HSV family that contains the ICP10 gene. The term “FusOn-H2” as used herein refers to a HSV-2 mutant having a modified ICP10 polynucleotide encoding a polypeptide having ribonucleotide reductase activity, but lacking protein kinase activity as described herein. FusOn-H2 and the modified ICP10 polynucleotide are described in U.S. Pat. Nos. 8,986,672 and 10,039,796 and U.S. Patent Pub. No. 2015/0246086, which are hereby incorporated by reference.

The term “HSV-2 oncolytic virus” and “HSV-2 mutant” are used interchangeably herein.

The term “cell membrane fusion” and “fusion” as used herein refers to fusion of an outer membrane of at least two cells, such as two adjacent cells, for example.

The term “enhanced fusogenic activity” as used herein refers to an enhancement, increase, intensification, argumentation, amplification, or combination thereof of the cell membrane fusion.

The term “oncolytic” as used herein refers to a property of an agent that can result directly or indirectly, in the destruction of malignant cells. In a specific embodiment, this property comprises causing fusion of a malignant cell membrane to another membrane.

The term “vector” as used herein refers to a carrier nucleic acid molecule into which a nucleic acid sequence can be inserted for introduction into a cell where it can be replicated. The inserted nucleic acid sequence is referred to as “exogenous” either when it is foreign to the cell into which the vector is introduced or when it is homologous to a sequence in the cell but in a position within the host cell nucleic acid in which die sequence is ordinarily not found. A vector can be either a non-viral DNA vector or a viral vector. Viral vectors are encapsulated in viral proteins and capable of infecting cells. non-limiting examples of vectors include: a viral vector, a non-viral vector, a naked DNA expression vector, a plasmid, a cosmid, an artificial chromosome (e.g., YACS), a phage-vector, a DNA expression vector associated with a cationic condensing agent, a DNA expression vector encapsulated in a liposome, or a certain eukaryotic cell e.g., a producer cell. Unless stated otherwise, “vector” as used herein refers both a DNA vector and a viral vector. One of skill in the art would be well equipped to construct a vector through standard recombinant techniques. Generally, these include Sambrook et al., Molecular Cloning: A Laboratory Manual, 2nd Ed., Cold Spring Harbor Laboratory Press (1989) and the references cited therein. Virological considerations are also reviewed in Coen D. M, Molecular Genetics of Animal Viruses in Virology, 2nd Edition, B. N. Fields (editor), Raven Press, N.Y. (1990) and the references cited therein.

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

A “promoter” is a control sequence that is a region of a nucleic acid sequence at which initiation and rate of transcription are controlled. It may contain genetic elements at which regulatory proteins and molecules may bind, such as RNA polymerase and other transcription factors to initiate or regulate the temporal and spatial transcription of a nucleic acid sequence. The phrases “operatively positioned,” “operably linked,” “under control,” and “under transcriptional control” mean that a promoter is in a correct functional location and/or orientation in relation to a nucleic acid sequence to control transcriptional initiation and/or expression of that sequence. Exemplary non-limiting promoters include: a constitutive promoter, a tissue-specific promoter, a tumor-specific promoter, or an endogenous promoter under the control of an exogenous inducible element.

The term “constitutive promoter” as used herein refers to a promoter that drives expression of a gene or polynucleotide in a continuous temporal manner throughout the cell cycle. A constitutive promoter may be cell or tissue-type specific as long as it operates in a continuous fashion throughout the cell cycle to drive the expression of the gene or polynucleotide with which it is associated. Exemplary non-limiting constitutive promoters include: the immediate early cytomegalovirus (CMV) promoter, SV40 early promoter, RSV LTR, Beta chicken actin promoter, and HSV TK promoter.

The term “enhancer” refers to a cis-acting regulatory sequence involved in the control of transcriptional activation of a nucleic acid sequence.

The phrase “modified ICP10 polynucleotide” refers to an ICP10 polynucleotide that encodes for an ICP10 polypeptide that has ribonucleotide reductase (RR) activity, but lacks protein kinase activity.

The phrase “ribonucleotide reductase activity” refers to ability of the C-terminal domain of the polypeptide encoded by an ICP10 polynucleotide to generate sufficient deoxynucleotide triphosphates (dNTPs) required for viral replication.

The phrase “protein kinase activity” refers to the ability of the amino-terminal domain of the polypeptide encoded by an ICP10 polynucleotide to phosphorylate serine and threonine residues capable of activating the Ras/MEK/MAPK pathway.

The term “effective” or “therapeutically effective” as used herein refers to suppressing or inhibiting an exacerbation in symptoms, inhibiting, suppressing, or preventing onset of a disease, inhibiting, suppressing, or preventing spread of disease, amelioration of at least one symptom of disease, or a combination thereof.

The term “patient” or “subject” refers to a mammal such as human or a domestic animal (e.g., a dog or cat). In a preferred embodiment, the patient or subject is a human.

The term “sera” or “serum” refers to the fluid from blood that remains when hematocytes and clotting proteins are removed.

The term “anti-cancer agent” as used herein refers to an agent that is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The phrases “pharmaceutically” or “pharmacologically acceptable” as used herein refer to molecular entities and compositions that do not produce an adverse, allergic or other untoward reaction when administered to an animal, or human, as appropriate. The phrase “pharmaceutically acceptable carrier” includes any and all solvents, dispersion media, coatings, antibacterial and antifungal agents, isotonic and absorption delaying agents and the like.

The term “unit dose” refers to a physically discrete unit suitable for use in a subject, each unit containing a predetermined quantity of the therapeutic composition calculated to produce the desired response in association with its administration, i.e., the appropriate route and treatment regimen.

### Introduction

Viruses can only replicate inside living cells and their replication usually requires activation of certain cellular signaling pathways. Many viruses have acquired various strategies during their evolution to activate these signaling pathways to benefit their replication. The large subunit of herpes simplex virus type 2 (HSV-2) ribonucleotide reductase (ICP10 or RR1) comprises a unique amino-terminal domain that has serine/threonine protein kinase (PK) activity. This PK activity has been found to activate the cellular Ras/MEK/MAPK pathway (Smith, et al., (2000) J Virol 74(22): 10417-29). Consequently, it has been reported that deletion of this PK domain (ICP10 PK) from the ribonucleotide reductase gene severely compromises the ability of the virus to replicate in cells, such as those where there is no preexisting activated Ras signaling pathway (Smith, et al., (1998) J Virol. 72(11):9131-9141).

When the PK domain of HSV-2 is replaced and/or modified such that protein encoded by the modified ICP10 gene has ribonucleotide reductase activity, but lacks protein kinase activity, the virus selectively replicates in and destroys tumor cells (at least tumor cells in which the Ras signaling pathway is constitutively activated due to tumorigenesis). Furthermore, modification of the ICP10 polynucleotide as described herein renders the virus intrinsically fusogenic, i.e., infection of tumor cells with the virus induces widespread cell membrane fusion (syncytial formation). This property increases the destructive power of the virus against tumor cells. Furthermore, in vivo studies show that this virus is extremely safe for either local or systemic administration.

In some embodiments of the invention, the modification of the PK domain comprises insertion of a reporter gene, such as that expressing the green fluorescent gene, and/or replacement of the native promoter gene with a constitutive promoter, such as the immediate early cytomegalovirus promoter.

In some embodiments, the HSV-2 is genetically engineered either by inserting a second polynucleotide into the polynucleotide encoding the protein kinase activity domain of the ICP10 gene, or by replacing a portion of the protein kinase domain with a second polynucleotide such that the polypeptide encoded by the modified polynucleotide has ribonucleotide reductase activity, but lacks protein kinase activity. For example, the second polynucleotide may encode a glycoprotein, such as a fusogenic membrane glycoprotein. A preferred glycoprotein for use within the scope of the present invention is a truncated form of gibbon ape leukemia virus envelope fusogenic membrane glycoprotein (GALV.fus). In certain aspects of the invention, expression of GALV.fus in the context of the oncolytic virus of the present invention significantly enhances the anti-tumor effect of the virus.

In some embodiments, the modified HSV-2 of the invention comprises a mutation, such as a deletion, in ICP10 that provides cell fusogenic properties to the virus. Such a mutation may be generated randomly during the virus screening or obtained from nature, and a pool of potential candidates for having cell fusogenic properties is then assayed for the function by means described herein and/or known in the art. A mutation leading to the fusogenic phenotype may be a point mutation, a frame shift, an inversion, a deletion, a splicing error mutation, a post-transcriptional processing mutation, over expression of certain viral glycoproteins, a combination thereof, and so forth. The mutation may be identified by sequencing the particular HSV-2 and comparing it to a known wild type sequence.

The modified HSV-2 of the present invention is useful for the treatment of malignant cells, such as, for example, to inhibit their spread, decrease or inhibit their division, eradicate them, prevent their generation or proliferation, or a combination thereof. The malignant cells may be from any form of cancer, such as a solid tumor, although other forms are also treatable. The modified HSV-2 of the present invention is useful for the treatment of lung, liver, prostate, ovarian, breast, brain, pancreatic, testicular, colon, head and neck, melanoma, and other types of malignancies. The invention is useful for treating malignant cells at any stage of a cancer disease, including metastatic stages of the disease. The invention may be utilized as a stand-alone therapy or in conjunction with another means of therapy, including chemotherapy, surgery, or radiation.

### Modified ICP10 Polynucleotide

The present invention describes a HSV-2 mutant having a modified ICP10 polynucleotide, wherein the modified ICP10 polynucleotide encodes for a polypeptide that has ribonucleotide reductase activity, but lacks protein kinase (PK) activity. The ICP10 polynucleotide may be modified either by deleting at least some of the sequence required for encoding a functional PK domain, or replacing at least part of the sequence encoding the PK domain with a second polynucleotide. One of skill in the art will recognize that any suitable method can be used for generating the modified ICP10 polynucleotide, including mutagenesis, polymerase chain reaction, homologous recombination, or any other genetic engineering technique known to a person of skill in the art.

### Mutagenesis

In specific embodiments of the invention, an ICP10 sequence of an HSV-2 virus, is mutated, such as by deletion, using any of a variety of standard mutagenic procedures. Mutation can involve modification of a nucleotide sequence, a single gene, or blocks of genes. A mutation may involve a single nucleotide (such as a point mutation, which involves the removal, addition or substitution of a single nucleotide base within a DNA sequence) or it may involve the insertion or deletion of large numbers of nucleotides. Mutations can arise spontaneously as a result of events such as errors in the fidelity of DNA replication, or induced following exposure to chemical or physical mutagens. A mutation can also be site-directed through the use of particular targeting methods that are well known to persons of skill in the art.

### Genetic Recombination

In other embodiments of the invention, the ICP10 polynucleotide is modified using genetic recombination techniques to delete or replace at least part of the sequence encoding for the PK domain. The region of the PK domain that is deleted/replaced may be any suitable region so long as the polypeptide encoded by the modified ICP10 polynucleotide retains ribonucleotide reductase activity and lacks protein kinase activity. In certain embodiments, though, the modification to the PK domain affects one or more of the eight PK catalytic motifs (amino acid residues 106-445, although the PK activity may be considered amino acid residues 1-445), and/or the transmembrane (TM) region, and/or the invariant Lys (Lys 176). An exemplary wild-type ICP10 polypeptide sequence is provided in SEQ ID NO: 15 (National Center for Biotechnology Information's GenBank database Accession No. 1813262A). An exemplary wild-type polynucleotide that encodes an ICP10 polypeptide is provided in SEQ ID NO: 17.

In certain embodiments, the ICP10 polynucleotide is modified by merely deleting a portion of the sequence encoding the PK domain that is necessary for PK activity. An exemplary ICP10 polynucleotide lacking at least some sequence that encodes a PK domain is provided in SEQ ID NO: 18. In another exemplary embodiment, ICP10 polynucleotide is modified such that the PK domain is deleted in its entirety, as provided in SEQ ID NO: 19. Both SEQ ID NO: 18 and SEQ ID NO: 19 are suitable for use in generating a HSV-2 mutant as described herein, as both sequences encode for polypeptides that have ribonucleotide reductase activity, but lack protein kinase activity. In certain embodiments of the invention, the modified ICP10 polynucleotide disclosed in SEQ ID NO: 18 or SEQ ID NO: 19 may be under the control of the endogenous HSV-2 promoter, or operably linked to a constitutive promoter, such as the immediate early cytomegalovirus promoter described in SEQ ID NO: 20.

In still other embodiments of the invention, the ICP10 polynucleotide is modified by replacing at least part of the sequence encoding the PK domain with a second polynucleotide, such as green fluorescent protein, which is placed in frame with the sequence encoding the RR domain of the ICP10 polynucleotide. This construct can be either under control of the endogenous HSV-2 promoter, or under the control of a constitutive promoter such as the CMV promoter (SEQ ID NO: 20).

In another aspect of the invention, the polynucleotide that replaces at least part of the protein kinase activity domain of the endogenous ICP10 in HSV-2 can encode at least a fusogenic portion of a cell membrane fusion-inducing polypeptide, such as a viral fusogenic membrane glycoprotein (FMG). The polypeptide is preferably capable of inducing cell membrane fusion at a substantially neutral pH (such as about pH 6-8), for example.

In particular embodiments, the FMG comprises at least a fusogenic domain from a C-type retrovirus envelope protein, such as MLV (as an example, SEQ ID NO: 6) or GALV (as an example, SEQ ID NO: 5). A retroviral envelope protein having a deletion of some, most, or all of the cytoplasmic domain is useful, because such manipulation results in hyperfusogenic activity for human cells. Particular modifications are introduced, in some embodiments, into viral membrane glycoproteins to enhance their function to induce cell membrane fusion. For example, truncation of the cytoplasmic domains of a number of retroviral and herpes virus glycoproteins has been shown to increase their fusion activity, sometimes with a simultaneous reduction in the efficiency with which they are incorporated into virions (Rein et al., (1994) J Virol 68(3): 1773-81).

Some examples of cell membrane fusing polypeptides include measles virus fusion protein (SEQ ID NO: 7), the HIV gp160 (SEQ ID NO: 8) and SIV gp160 (SEQ ID NO: 9) proteins, the retroviral Env protein (SEQ ID NO: 10), the Ebola virus Gp (SEQ ID NO: 11), and the influenza virus haemagglutinin (SEQ ID NO: 12).

In other embodiments, a second functional polynucleotide may be either inserted into the PK domain, or used to replace part or all of the PK domain. This second functional polynucleotide may encode for an immunomodulatory or other therapeutic agent. It is contemplated that these additional agents will affect the upregulation of cell surface receptors and GAP junctions, cytostatic and differentiation agents, inhibit cell adhesion, or increase the sensitivity of the malignant cells to apoptosis. Exemplary, non-limiting examples of polynucleotides encoding for immunomodulatory or other therapeutic agents include tumor necrosis factor; interferon, alpha, beta, gamma; interleukin-2 (IL-2), IL-12, granulocyte macrophage-colony stimulating factor (GM-CSF), F42K, MIP-1, MCP-1, RANTES, Herpes Simplex Virus-thymidine kinase (HSV-tk), cytosine deaminase, and caspase-3.

In still other embodiments of the invention, the ICP10 polynucleotide is modified by insertion of a polynucleotide encoding a reporter protein. Exemplary non-limiting polynucleotides encoding for reporter proteins include green fluorescent protein, enhanced green fluorescent protein, β-galactosidase, luciferase, and HSV-tk.

### Ribonucleotide Reductase Activity Assay

The biologic activity of RR can be detected as previously described (Averett, et al., J. Biol. Chem. 258:9831-9838 (1983) and Smith et al., J. Virol. 72:9131-9141 (1998)) with the following modifications. BHK cells are initially grown to confluence in complete GMEM (containing 10% FBS) and then incubated for three days in 0.5% FBS EMEM, followed by infection with 20 pfu of wild-type HSV, HSV-2 mutant, or mock infection. The cells are harvested 20 hours post infection, resuspended in 500 μl HD buffer [100 mM HEPES buffer (pH 7.6), 2 mM dithiothreitol (DTT)] and incubated on ice for 15 minutes before a 30 second sonication. Cell debris is cleared by centrifugation (16,000 g, 20 minutes, 4° C.) and the supernatant is precipitated with crystalline ammonium sulfate at 45% saturation (0.258 g/ml). After a second centrifugation (16,000 g, 30 minutes), the pellets are dissolved in 100 μl HD buffer, from which 50 μl is taken to mix with an equal volume of 2× reaction buffer (400 mM HEPES buffer (pH 8.0), 20 mM DTT and 0.02 mM [³H]-CDP (24 Ci/mmol, Amersham, Chicago, Ill.). The reaction is terminated by the addition of 100 mM hydroxyurea with 10 mM EDTA (pH 8.0) and boiling for 3 minutes. Then 1 ml of Crotalux atrox venom (Sigma, St. Louis, Mo.) is added and incubated for 30 minutes at 37° C., followed by another 3 minute boiling. The solution is then passed through a 0.5 ml Dowex-1 borate column, and samples eluted with 2 ml water and collected in four elution fractions for scintillation counting after mixing with Biofluor (New England Nuclear, Boston, Mass.). Ribonucleotide reductase activity is expressed as units/mg protein where 1 unit represents the conversion of 1 nmol [³H]CDP to dCDP/hr/mg protein.

### Protein Kinase Activity Assay

To determine whether the modified ICP10 polynucleotide encodes a polypeptide that lacks protein kinase activity, extracts of cells infected with HSV-2 having a modified ICP10 polynucleotide or wild-type HSV-2 (moi=200, 16 hours post infection) are immunopercipitated with anti LA-1 antibody and subjected to PK assays as described in Chung et al. J. Virol. 63:3389-3398, 1998 and U.S. Pat. No. 6,013,265. Generally, immunopercipitates of cell extracts are normalized for protein concentration using a BCA protein assay kit (PIERCE, Rockford Ill.) washed with TS buffer containing 20 mM Tris-HCL (pH 7.4), 0.15 M NaCl, suspended in 50 μl kinase reaction buffer consisting of 20 mM Tris-HCL (pH 7.4) 5 mM MgCl2, 2 mM Mn C12, 10 μCi [³²p] ATP (3000 Ci/mmol, DuPont, New England Research Prod.) and incubated at 30° C. for 15 minutes. The beads are washed once with 1 ml TS buffer, resuspended in 100 μl denaturing solution and boiled for 5 minutes. Proteins are then resolved by SDS-PAGE on a 7% polyacrylamide gel. Proteins are then electrotransferred onto nitrocellulose membranes as previously described (see, Aurelian et. al., Cancer Cells 7:187-191 1989) and immunoblotted by incubation with specific antibodies followed by protein A-peroxidase (Sigma, St. Louis, Mo.) for 1 hour at room temperature. Detection can be made with ECL reagents (Amersham, Chicago, Ill.) as described in Smith et al., Virol. 200:598-612, (1994).

### Vector Construction

The present invention is directed to an HSV-2 vector comprising a replacement or deletion of at least part of an ICP10 sequence, such that the protein encoded for by the modified ICP10 polynucleotide has ribonucleotide reductase activity, but lacks protein kinase activity, and in specific embodiments further comprising a regulatory sequence, such as a constitutive promoter. In some embodiments, the composition is a naked (non-viral) DNA vector comprising the modified ICP10 gene, and in other embodiments, the composition is a recombinant HSV-2 having the modified ICP10 gene. Both the naked DNA vector, and the recombinant virus can be further comprised of some or all of the following components.

### Vectors

Vectors, as defined supra, include but are not limited to plasmids, cosmids, viruses (bacteriophage, animal viruses, and plant viruses), and artificial chromosomes (e.g., YACs). Methods for the construction of engineered viruses and DNA vectors are known in the art. Generally these include Sambrook et al., Molecular Cloning: A Laboratory Manual, 2nd Ed., Cold Spring Harbor Laboratory Press (1989) and the references cited therein. Virological considerations are also reviewed in Coen D. M, Molecular Genetics of Animal Viruses in Virology, 2.sup.nd Edition, B. N. Fields (editor), Raven Press, N.Y. (1990) and the references cited therein.

Expression vectors can contain a variety of “control sequences,” which refer to nucleic acid sequences necessary for the transcription and possibly translation of an operably linked coding sequence in a particular host cell. In addition to control sequences that govern transcription and translation, DNA vectors, expression vectors, and viruses may contain nucleic acid sequences that serve other functions as well and are described infra.

### 1. Promoters and Enhancers

A promoter generally comprises a sequence that functions to position the start site for RNA synthesis. The best-known example of this is the TATA box, but in some promoters lacking a TATA box (e.g., the promoter for the mammalian terminal deoxynucleotidyl transferase gene and the promoter for the SV40 late genes) a discrete element overlying the start site itself helps to fix the place of initiation. Additional promoter elements regulate the frequency of transcriptional initiation. Typically, these are located in the region 30 to 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an enhancer.

A promoter may be one naturally associated with a nucleic acid sequence, as may be obtained by isolating the 5′ non-coding sequences located upstream of the coding segment and/or exon. Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the R lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR, in connection with the compositions disclosed herein (see U.S. Pat. Nos. 4,683,202 and 5,928,906). Furthermore, it is contemplated that control sequences, which direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination may be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art. Non-limiting examples of such regions include the human LIMK2 gene (Nomoto et al. (1999) Gene 236(2):259-271), the somatostatin receptor-2 gene (Kraus et al., (1998) FEBS Lett. 428(3): 165-170), murine epididymal retinoic acid-binding gene (Lareyre et al., (1999) J. Biol. Chem. 274(12):8282-8290), human CD4 (Zhao-Emonet et al., (1998) Biochem. Biophys. Acta, 1442(2-3):109-119), mouse α-2 (XI) collagen (Tsumaki, et al., (1998), J. Biol. Chem. 273(36):22861-4) D 1 A dopamine receptor gene (Lee, et al., (1997), DNA Cell Biol. 16(11):1267-1275) insulin-like growth factor II (Vu et al., (1997) Biophys Biochem Res. Comm. 233(1):221-226) and human platelet endothelial cell adhesion molecule-1 (Almendro et al., (1996) J. Immunol. 157(12):5411-5421).

### 2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819).

### 3. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently. Thus, in other embodiments involving eukaryotes, it is contemplated that the terminator comprise a signal for the cleavage of the RNA, and that the terminator signal promote polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

### 4. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, both of which are convenient and known to function well in various target cells. Polyadenylation may increase the stability of the transcript or may facilitate cytoplasmic transport.

### 5. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with fluorescence activated cell sorting (FACS) analysis. The marker used is not believed to be important, so long as it is capable of being expressed simultaneously with the nucleic acid encoding a gene product. Further examples of selectable and screenable markers are well known to one of skill in the art.

The vector is introduced to the initially infected cell by suitable methods. Such methods for nucleic acid delivery for transformation of an organelle, a cell, a tissue or an organism for use with the current invention are believed to include virtually any method by which a nucleic acid (e.g., HSV vector) can be introduced into an organelle, a cell, a tissue or an organism, as described herein or as would be known to one of ordinary skill in the art. Non-limiting exemplary methods include: direct delivery of DNA by ex vivo transfection; injection (U.S. Pat. Nos. 5,994,624, 5,981,274, 5,945,100, 5,780,448, 5,736,524, 5,702,932, 5,656,610, 5,589,466 and 5,580,859); microinjection (U.S. Pat. No. 5,789,215); electroporation (U.S. Pat. No. 5,384,253); calcium phosphate precipitation; DEAE dextran followed by polyethylene glycol; direct sonic loading; liposome mediated transfection; receptor-mediated transfection; microprojectile bombardment (PCT Application Nos. WO 94/09699 and 95/06128; U.S. Pat. Nos. 5,610,042; 5,322,783 5,563,055, 5,550,318, 5,538,877 and 5,538,880); agitation with silicon carbide fibers (U.S. Pat. Nos. 5,302,523 and 5,464,765); Agrobacterium mediated transformation (U.S. Pat. Nos. 5,591,616 and 5,563,055); PEG mediated transformation of protoplasts (U.S. Pat. Nos. 4,684,611 and 4,952,500); desiccation/inhibition mediated DNA uptake, and any combination of these methods, or other methods known to persons of skill in the art. The composition can also be delivered to a cell in a mammal by administering it systemically, such as intravenously, in a pharmaceutically acceptable excipient.

### Methods of DNA Vector Delivery to Cells

### 1. Ex Vivo Transformation

Methods for transfecting cells and tissues removed from an organism in an ex vivo setting are known to those of skill in the art. Thus, it is contemplated in the present invention that cells or tissues may be removed and transfected ex vivo using the nucleic acids and compositions described herein. In particular aspects, the transplanted cells or tissues may be placed into an organism. In some embodiments, a nucleic acid is expressed in the transplanted cell or tissue.

### 2. Injection

In certain embodiments, a nucleic acid may be delivered to an organelle, a cell, a tissue or an organism via one or more injections (i.e., a needle injection), such as, for example, subcutaneously, intradermally, intramuscularly, intravenously, intraperitoneally, etc. Methods of injection are well known to those of ordinary skill in the art (e.g., injection of a composition comprising a saline solution). Further embodiments of the present invention include the introduction of a nucleic acid by direct microinjection. The amount of composition of the present invention used may vary upon the nature of the cell, tissue or organism affected.

### 3. Electroporation

In certain embodiments of the present invention, a nucleic acid is introduced into an organelle, a cell, a tissue or an organism via electroporation. Electroporation involves the exposure of a suspension of cells and DNA to a high voltage electric discharge. In some variants of this method, certain cell wall degrading enzymes, such as pectin degrading enzymes, are employed to render the target recipient cells more susceptible to transformation by electroporation than untreated cells (U.S. Pat. No. 5,384,253). Alternatively, recipient cells can be made more susceptible to transformation by mechanical wounding.

### 4. Liposome Mediated Transfection

In a further embodiment of the invention, a composition as described herein, such as a vector having a modified ICP10 polynucleotide, may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self rearrangement before the formation of closed structures and entrap water and dissolved solutes between the lipid bilayers. Also contemplated is an nucleic acid complexed with Lipofectamine (Gibco BRL) or Superfect (Qiagen).

In certain embodiments of the invention, a liposome may be complexed with a hemagglutinatin virus (HVJ). This has been shown to facilitate fusion with the cell membrane and promote cell entry of liposome encapsulated DNA (Kaneda et al., (1989) Science 20; 243(4889):375-8). In other embodiments, a liposome may be complexed or employed in conjunction with nuclear non histone chromosomal proteins (HMG1) (Kato et al., (1991) J Biol Chem. (1991) February 25; 266(6):3361-4). In yet further embodiments, a liposome may be complexed or employed in conjunction with both HVJ and HMG 1. In other embodiments, a delivery vehicle may comprise a ligand and a liposome.

### 5. Receptor Mediated Transfection

A nucleic acid may be delivered to a target cell via receptor mediated delivery vehicles. This approach takes advantage of the selective uptake of macromolecules by receptor mediated endocytosis. In view of the cell type specific distribution of various receptors, this delivery method adds another degree of specificity to the present invention.

In certain embodiments, the receptor mediated gene targeting vehicle comprises a receptor specific ligand and a nucleic acid binding agent. Other embodiments comprise a receptor specific ligand to which the nucleic acid to be delivered has been operatively attached. Several ligands have been used for receptor mediated gene transfer including the epidermal growth factor (EGF), which has been used to deliver genes to squamous carcinoma cells as described in European Patent No. EPO 0 273 085.

In other embodiments, a nucleic acid delivery vehicle component of a cell specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell.

In still further embodiments, the nucleic acid delivery vehicle component of a targeted delivery vehicle may be a liposome itself, which will preferably comprise one or more lipids or glycoproteins that direct cell specific binding. For example, lactosyl ceramide, a galactose terminal asialganglioside, has been incorporated into liposomes and an increase in the uptake of the insulin gene by hepatocytes has been observed (Nicolau et al., (1987) Methods Enzymol. 149:157-76). It is contemplated that the tissue specific transforming constructs of the present invention can be specifically delivered into a target cell in a similar manner.

### 6. Microprojectile Bombardment

Microprojectile bombardment techniques can be used to introduce a nucleic acid into at least one, organelle, cell, tissue or organism (U.S. Pat. Nos. 5,550,318; 5,538,880; 5,610,042; and PCT Application No. WO 94/09699). This method depends on the ability to accelerate microprojectiles that are either coated with DNA or contain DNA, to a high velocity allowing them to pierce cell membranes and enter cells without killing them. The microprojectiles may be comprised of any biologically inert substance, such as tungsten, platinum, or gold. For the bombardment, cells in suspension are concentrated on filters or solid culture medium. Alternatively, immature embryos or other target cells may be arranged on solid culture medium. The cells to be bombarded are positioned at an appropriate distance below the microprojectile bombardment device on a stopping plate. A wide variety of microprojectile bombardment techniques useful for practice with the current invention will be known to persons of skill in the art.

### Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells that do not contain a recombinantly introduced nucleic acid.

A tissue may comprise a host cell or cells to be transformed with a cell membrane fusion-generating HSV-2 mutant. The tissue may be part or separated from an organism. In certain embodiments, a tissue may comprise, but is not limited to, adipocytes, alveolar, ameloblasts, neural, basal cells, blood (e.g., lymphocytes), blood vessel, bone, bone marrow, glial cell, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, small intestine, spleen, stem cell, stomach, testes, and all cancers thereof.

In certain embodiments, the host cell or tissue may be comprised in at least one organism. In certain embodiments, the organism may be, but is not limited to, a prokaryote (e.g., a eubacteria, an archaea) or a eukaryote, as would be understood by one of ordinary skill in the art.

Numerous cell lines and cultures are available for use as a host cell, and are commercially available through organizations such as the American Type Culture Collection (ATCC). An appropriate host can be determined by one of skill in the art based on the vector backbone and the desired result. Exemplary non-limiting cell types available for vector replication and/or expression include bacteria, such as E. coli (e.g., E. coli strains RR1, LE392, B, X 1776 (ATCC No. 31537), W3110, F, lambda, DH5α, JM109, and KC8); bacilli e.g., Bacillus subtilis; other enterobacteriaceae e.g., Salmonella typhimurium, Serratia marcescens, as well as a number of commercially available bacterial hosts and competent cells such as SURE® Competent Cells and SOLOPACK™ Gold Cells (STRATAGENE®, La Jolla, Calif.). Non-limiting examples of eukaryotic host cells for replication and/or expression of a vector include, HeLa, NIH3T3, Jurkat, 293, Cos, CHO, Saos, and PC12.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### Viral Vector Packaging and Propagation

### 1. Viral Packaging

In specific embodiments of the present invention, after the ICP10 gene has been modified, it is inserted into the virus through homologous recombination. Typically, this is done by co-transfecting the plasmid DNA containing the modified ICP10 gene with purified HSV-2 genomic DNA into Vero cells using Lipofectamine. The recombinant virus is then identified (typically by screening the virus plaques for the presence of a selectable marker) and selecting plaques containing the modified ICP10 polynucleotide. The selected recombinant virus is then characterized in vitro to confirm that the modified ICP10 gene has been correctly inserted into the HSV-2 genome to replace the original ICP10 gene.

### 2. Preparation of Viral Stocks

Once the recombinant HSV-2 mutant virus has been selected, viral stocks can be prepared as follows. Vero cells are grown in 10% fetal bovine serum (FBS) and infected with 0.01 plaque forming units (pfu) per cell. Viruses are then harvested from the cells 2 days later by repeated freezing and thawing and sonication. The harvested virus is then purified as described (Nakamori, et al., (2003) Clinical Cancer Res. 9(7):2727-2733). The purified virus is then titered, aliquoted and stored at −80° C. until use.

### Protein Expression Systems

Protein expression systems may be utilized in the generation of DNA vector compositions of the present invention for example, to express the polypeptide encoded by the modified ICP10 polynucleotide for functional studies. Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236 and is commercially available (e.g., CLONTECH, Inc. Mountain View, Calif.).

Other examples of commercially available expression systems include an inducible mammalian expression system, which involves a synthetic ecdysone-inducible receptor, or a pET expression system, or an E. coli expression system (STRATAGENE, LaJolla, Calif); A tetracycline-regulated expression system, an inducible mammalian expression system that uses the full-length CMV promoter or a yeast expression system designed for high-level production of recombinant proteins in the methylotrophic yeast Pichia methanolica (INVITROGEN, Carlsbad, Calif.).

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analysis, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

### Functional Roles of a HSV-2 Mutant

A HSV-2 mutant as described herein displays multiple functional roles as an oncolytic agent. For example, the virus can destroy tumor cells by lysis, as well as by syncytial formation, and induction of apoptosis in both infected cells as well as by-stander cells. Furthermore, tumor destruction by the HSV-2 mutant induces a potent anti-tumor immune response that further contributes to the therapeutic efficacy of the mutant virus as an oncolytic agent for the treatment of malignant disease.

The HSV-2 mutant virus displays selective replication in cycling, but not non-cycling cells. The mutant HSV-2, lacking protein kinase activity, shows at least a 40-fold decrease in growth in non-cycling cells as compared to growth in cycling cells. In contrast, the wild-type HSV-2 is only marginally affected in its growth characteristics between cycling and non-cycling cells. Therefore, the HSV-2 mutant as described herein is well suited for use as an oncolytic agent in cycling cells having an activated Ras pathway, such as tumor cells.

In addition to the lytic and fusogenic activities, the HSV-2 mutant also has potent apoptotic inducing activity and is capable of inducing a potent anti-tumor immune response. In an in vitro setting, the HSV-2 mutant can induce apoptosis in cells infected with the virus as well as non-infected by-stander cells that surround the infected cells. Furthermore, HSV-2 mutant is effective at inducing apoptosis of tumor cells in vivo. Not only are the compositions described herein more effective at killing tumor cells than other oncolytic viruses, the HSV-2 mutant displays a strong therapeutic effect against primary and metastatic tumor in vivo by induction of a strong anti-tumor immune response. The adoptive transferred CTL from FusOn-H2 treated mice can inhibit the growth of the original tumor and effectively prevent the metastases developing.

Apoptosis, or programmed cell death, is an essential process for normal embryonic development, maintaining homeostasis in adult tissues, and suppressing carcinogenesis. In some embodiments of the invention, the modified HSV-2 is a potent inducer of apoptosis in tumor cells infected with the virus, and in non-infected by-stander tumor cells. For example, in a particular embodiment tumor cells were infected with an HSV-2 construct in which parts of the protein kinase domain of the ICP10 gene was replaced with a gene encoding the green fluorescent protein (GFP). Infected cells could be identified under a fluorescent microscope by visualizing the GFP, and cells undergoing apoptosis were identified as evidenced by their chromatin condensation. The ratio of cells showing chromatin condensation to GFP expression was 2.6:1, suggesting that there was a substantial number of tumor cells undergoing apoptosis, that were not infected with the modified HSV-2.

Strong anti-tumor immune responses are useful in combating malignant disease. The HSV-2 mutant described herein is capable of inducing a potent antitumor immune response against primary and metastatic tumors in vivo. In a particular embodiment, the mutant HSV-2 (FusOn-H2) selectively replicated in and lysed tumor cells in a mouse mammary tumor model using the 4T1 mouse mammary tumor cell line, and showed a strong therapeutic effect against primary and metastatic tumor in vivo by induction of strong antitumor immune response. Specifically, adoptive transferred cytotoxic T lymphocytes (CTL) from FusOn-H2 treated mice can inhibit growth of the original tumor and effectively prevent metastasis in mice not treated with FusOn-H2.

### Pharmaceutical Compositions and Routes of Administration

Compositions of the present invention can be administered as a pharmaceutical composition comprising either a recombinant HSV-2 mutant having a modified ICP10 gene, or as a naked (non-viral) DNA vector having a modified ICP10 gene, as described herein. The compositions of the present invention include classic pharmaceutical preparations. In general, the compositions of the present invention can be administered as pharmacological agents by dissolving or dispersing the composition in a pharmaceutically acceptable carrier or aqueous medium. The use of such media and agents for pharmaceutical active substances is well known in the art. Except insofar as any conventional media or agent is incompatible with the compositions of the invention, its use in a therapeutic composition is contemplated. Supplementary active ingredients, such as other anti-disease agents, can also be incorporated into the pharmaceutical composition. Administration of the composition will be via any common route so long as the target cell is available via that route. Exemplary administration routes include oral, nasal, buccal, rectal, vaginal or topical. Alternatively, administration may be by orthotopic, intradermal, subcutaneous, intramuscular, intraperitoneal, intravenous, or direct intratumoral injection. The pharmaceutical formulations, dosages and routes of administration for the compositions of the present invention are described infra.

### Pharmaceutical Formulation of HSV-2 Mutant

The mutant viral composition of the present invention can be prepared as a pharmacologically acceptable formulation. Typically, the mutant virus is mixed with an excipient which is pharmaceutically acceptable and compatible with the virus. Suitable excipients are, for example, water, saline, dextrose, glycerol, ethanol, or the like and combinations thereof. In addition, if desired, the preparation may contain minor amounts of auxiliary substances such as wetting or emulsifying agents, pH-buffering agents, adjuvants or immunopotentiators, which enhance the effectiveness of the viral mutant (See, Remington's Pharmaceutical Sciences, Gennaro, A. R. et al., eds., Mack Publishing Co., pub., 18th ed., 1990). For example, a typical pharmaceutically acceptable carrier for injection purposes may comprise from 50 mg up to about 100 mg of human serum albumin per milliliter of phosphate buffered saline. Additional non-limiting exemplary non-aqueous solvents suitable for use in the formulation of a pharmacologically acceptable composition include propylene glycol, polyethylene glycol, vegetable oil, sesame oil, peanut oil and injectable organic esters such as ethyloleate. Exemplary non-limiting aqueous carriers include water, aqueous solutions, saline solutions, parenteral vehicles such as sodium chloride, Ringer's dextrose, etc. Intravenous vehicles include fluid and nutrient replenishers. Determining the pH and exact concentration of the various components of the pharmaceutical composition is routine and within the knowledge of one of ordinary skill in the art (See Goodman and Gilman's The Pharmacological Basis for Therapeutics, Gilman, A. G. et al., eds., Pergamon Press, pub., 8th ed., 1990).

Sterile injectable solutions are prepared by incorporating the active compounds in the required amount in the appropriate solvent with various other sterile ingredients as required and described above. Generally, dispersions are prepared by incorporating the various sterilized active ingredients into a sterile vehicle which contains the basic dispersion medium and the required other ingredients as described above.

### Routes and Dosages for Administration of HSV-2 Mutant

The mutant viral composition may be delivered by any route that provides access to the target tissue. Exemplary non-limiting routes of administration may include oral, nasal, buccal, rectal, vaginal topical, or by injection (including orthotopic, intradermal, subcutaneous, intramuscular, intraperitoneal, intravenous, or direct intratumoral injection). Typically, the viral mutant would be prepared as an injectable, either as a liquid solution or a suspension; a solid form suitable for solution in, or suspension in, liquid prior to injection may also be prepared. The preparation also may be emulsified.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 ml of isotonic NaCl solution and either added to 1000 ml of hypodermolysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Those of skill in the art will recognize that the best treatment regimens for using a composition of the present invention to provide therapy can be straightforwardly determined. This is not a question of experimentation, but rather one of optimization, which is routinely conducted in the medical arts. For example, in vivo studies in mice provide a starting point from which to begin to optimize the dosage and delivery regimes. The frequency of injection may initially be once a week. However, this frequency might be optimally adjusted from one day to every two weeks to monthly, depending upon the results obtained from the initial clinical trials and the needs of a particular patient. Human dosage amounts can initially be determined by extrapolating from the amount of composition used in mice.

### Dosages

The amount of viral vector delivered will depend on several factors including number of treatments, subject to be treated, capacity of the subjects immune system to synthesize anti-viral antibodies, the target tissue to be destroyed, and the degree of protection desired. The precise amount of viral composition to be administered depends on the judgment of the practitioner and is peculiar to each individual. However, suitable dosage ranges from 10⁵ plaque forming units (pfu) to 10¹⁰ pfu. In certain embodiments, the dosage of viral DNA may be about 10⁵, 10⁶, 10⁷, 10⁸, 10⁹, up to and including 10¹⁰ pfu.

### Non-Viral DNA Vector Formulation

In addition to the formulations described above for viral pharmaceutical formulation, the non-viral DNA vector can also be prepared as a sterile powder for the preparation of pharmacologically acceptable sterile solutions. Typical methods for preparation of sterile powder include vacuum-drying and freeze-drying techniques which yield a powder of the active ingredient plus any additional desired ingredient from a previously sterile-filtered solution thereof.

### Routes and Dosages for Administration of Non-Viral DNA Vector

Several methods for the delivery of non-viral vectors for the transfer of a polynucleotide of the present invention into a mammalian cell is contemplated. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection as discussed previously. Some of these techniques may be successfully adapted for in vivo or ex vivo use.

In some embodiments of the present invention, the expression vector may simply consist of naked recombinant DNA or plasmids comprising the polynucleotide. Transfer of the construct may be performed by any of the methods mentioned herein which physically or chemically permeabilize the cell membrane. This is particularly applicable for transfer in vitro, but it may be applied to in vivo use as well.

In other embodiments, the delivery vehicle may comprise a ligand and a liposome. For example, Nicolau et al., employed lactosyl-ceramide, a galactose-terminal asialganglioside, incorporated into liposomes and observed an increase in the uptake of the insulin gene by hepatocytes (Nicolau et al., (1987) Methods Enzymol. 149:157-76). Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor (as described in European Patent No. EP 0 273 085) and mannose can be used to target the mannose receptor on liver cells.

In certain embodiments, DNA transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissue.

### Dosages

In certain embodiments it is envisioned that the dosage may vary from between about 10³ pfu/kg body weight to about 10⁸ pfu/kg body weight. In certain embodiments, the dosage may be from about 10³, 10⁴, 10⁵, 10⁶, 10⁷, up to and including 10⁸ pfu/kg body weight. Of course, this dosage amount may be adjusted upward or downward, as is routinely done in such treatment protocols, depending on the results of the initial clinical trials and the needs of a particular patient.

### Combination Treatments

In order to increase the effectiveness of the methods and compositions of the present invention, it may be desirable to combine the methods and compositions disclosed herein with other anti-cancer agents. This process may involve contacting the cancer cell with a composition of the present invention in conjunction with at least one other anti-cancer agent. This may be achieved by contacting the cell with a single composition or pharmacological formulation that includes both agents, or by contacting the cell with two distinct compositions or formulations. Where two distinct formulations are used, the cancer cell may be contacted either by both formulations at the same time, or where one formulation precedes the other (e.g. where a composition of the present invention is administered either preceding or following administration of another anti-cancer agent) or any combination or repetitive cycle thereof. In embodiments where a composition of the present invention and the other agent are administered separately, one would generally ensure that a significant period of time did not expire between the time of each delivery, such that the composition of the present invention and the other agent would still be able to exert an advantageously combined effect on the cancer cell. This time interval between administration of the two formulations may range from minutes to weeks.

Non-limiting examples of anti-cancer agents that may be used in conjunction with the compositions or methods of the present invention may include chemotherapeutic agents (e.g., cisplatin (CDDP), carboplatin, procarbazine, mechlorethamine, cyclophosphamide, camptothecin, ifosfamide, melphalan, chlorambucil, busulfan, nitrosourea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristin, vinblastin and methotrexate, or any analog or derivative variant of the foregoing); radio-therapeutic agents (e.g., γ-rays, X-rays, microwaves and UV-irradiation, and/or the directed delivery of radioisotopes to tumor cells); immunotherapeutic and immunomodulatory agents; gene therapeutic agents; pro-apoptotic agents and other cell cycle regulating agents well known to persons of skill in the art.

Immunotherapy can also be used in conjunction with the compositions and methods described herein as a combination therapy for the treatment of malignant disease. Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells (e.g. cytotoxic T-cells or NK cells) to actually effect cell killing. The antibody also may be conjugated to a drug or toxin (e.g., a chemotherapeutic, radionuclide, ricin A chain, cholera toxin, pertussis toxin, etc.) and serve merely as a targeting agent. In some embodiments, the effector may be a lymphocyte carrying a surface molecule that interacts, either directly or indirectly, with a tumor cell target. In other embodiments, the tumor cell must bear some marker that is amenable to targeting. Non-limiting exemplary tumor markers suitable for targeting may include carcinoembryonic antigen (CEA), prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

In one preferred embodiment, checkpoint blockage immunotherapy is used in combination with the compositions and methods described herein. The checkpoint blockage immunotherapy may be conducted by administering a checkpoint blockage agent, such as a PD-L1 inhibitor (such as atezolizumab, avelumab, and durvalumab), PD-1 inhibitor (such as pembrolizumab, nivolumab, and cemiplimab), or a CTLA-4 inhibitor (such as ipilimumab), or by an adoptive T-cell transfer.

Gene therapy can also be used in conjunction with the compositions and methods described herein as a combination therapy for the treatment of malignant disease. Gene therapy as a combination treatment relies on the delivery and expression of a therapeutic gene, separate from the mutant HSV-2 described herein. The gene therapy can be administered either before, after, or at the same time as the HSV-2 mutant described herein. Exemplary non-limiting targets of gene therapy include immunomodulatory agents, agents that affect the up regulation of cell surface receptors and GAP junctions, cytostatic and differentiation agents, inhibitors of cell adhesion, or agents that induce or increase the sensitivity of target cells to apoptosis. Exemplary non-limiting immunomodulatory genes that can be used as part of gene therapy in combination with the present invention include tumor necrosis factor; interferon alpha, beta, and gamma; IL-2 and other cytokines; F42K and other cytokine analogs; Or MIP-1, MIP-1 beta, MCP-1, RANTES, and other chemokines.

An exemplary inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the Gl. The activity of this enzyme may be to phosphorylate Rb at late Gl. The activity of CDK4 is controlled by an activating subunit, D-type cyclin, and by an inhibitory subunit, the p16INK4 that specifically binds to and inhibits CDK4, and thus may regulate Rb phosphorylation. The p16INK4 gene belongs to a newly described class of CDK-inhibitory proteins that also includes p16B, p19, p21WAF1, and p27KIP1. Homozygous deletions and mutations of the p16INK4 gene are frequent in human tumor cell lines. Since the p16INK4 protein is a CDK4 inhibitor deletion of this gene may increase the activity of CDK4, resulting in hyperphosphorylation of the Rb protein. Other genes that may be employed with gene therapy to inhibit cellular proliferation include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, frns, trk, ret, gsp, hst, abl, E1A, p300, genes involved in angiogenesis (e.g., VEGF, FGF, thrombospondin, BAI-1, GDAIF, or their receptors) and MCC.

It is further contemplated that the up regulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases in intercellular signaling by elevating the number of GAP junctions would increase the anti-hyperproliferative effects on a neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### Development of Resistant Oncolytic Virus

A sophisticated process is described herein to generate an oncolytic virus based on FusOn-H2, that can escape all of the limiting factors in the bloodstream that reduce the effectiveness of oncolytic virotherapy. This disclosure also describes the design of the novel virus, FusOn-SD.

The native HSV-2 virus comprises an ICP10 polynucleotide (which may also be referred to as an RR1 polynucleotide) encoding a polypeptide having an amino-terminal domain with protein kinase (PK) activity, such as serine/threonine protein kinase activity and a c-terminal domain having ribonucleotide reductase activity. In particular aspects of the invention, the endogenous PK domain is modified such that the virus comprises selective replication activity in tumor cells (and therefore comprises activity to destroy tumor cells) and/or activity to render the virus fusogenic or have enhanced fusogenic activity, in that it comprises membrane fusion (syncytial formation) activity. In some embodiments, the ICP10 polynucleotide is modified by deleting at least part of the endogenous sequence encoding the protein kinase domain, such that the encoded polypeptide lacks protein kinase activity.

Viruses can only replicate inside living cells and their replication usually requires activation of certain cellular signaling pathways. Many viruses have acquired various strategies during their evolution to activate these signaling pathways to benefit their replication. The large subunit of herpes simplex virus type 2 (HSV-2) ribonucleotide reductase (ICP10 or RR1) comprises a unique amino-terminal domain that has serine/threonine protein kinase (PK) activity. This PK activity has been found to activate the cellular Ras/MEK/MAPK pathway (Smith, et al., (2000) J Virol 74(22): 10417-29). Consequently, it has been reported that deletion of this PK domain (ICP10 PK) from the ribonucleotide reductase gene severely compromises the ability of the virus to replicate in cells, such as those where there is no preexisting activated Ras signaling pathway (Smith, et al., (1998) J. Virol. 72(11):9131-9141).

In certain embodiments, an ICP10 sequence of an HSV-2 virus, is mutated, such as by deletion, using any of a variety of standard mutagenic procedures. Mutation can involve modification of a nucleotide sequence, a single gene, or blocks of genes. A mutation may involve a single nucleotide (such as a point mutation, which involves the removal, addition or substitution of a single nucleotide base within a DNA sequence) or it may involve the insertion or deletion of large numbers of nucleotides. Mutations can arise spontaneously as a result of events such as errors in the fidelity of DNA replication, or induced following exposure to chemical or physical mutagens. A mutation can also be site-directed through the use of particular targeting methods that are well known to persons of skill in the art.

The ICP10 polynucleotide is modified using genetic recombination techniques to delete or replace at least part of the sequence encoding for the PK domain. The region of the PK domain that is deleted/replaced may be any suitable region so long as the polypeptide encoded by the modified ICP10 polynucleotide retains ribonucleotide reductase activity and lacks protein kinase activity. The ICP10 polynucleotide may also be modified by merely deleting a portion of the sequence encoding the PK domain that is necessary for PK activity. The ICP10 polynucleotide may also be modified by replacing at least part of the sequence encoding the PK domain with a second polynucleotide, such as green fluorescent protein, which is placed in frame with the sequence encoding the RR domain of the ICP10 polynucleotide.

The polynucleotide that replaces at least part of the protein kinase activity domain of the endogenous ICP10 in HSV-2 can encode at least a fusogenic portion of a cell membrane fusion-inducing polypeptide, such as a viral fusogenic membrane glycoprotein (FMG). The polypeptide is preferably capable of inducing cell membrane fusion at a substantially neutral pH (such as about pH 6-8), for example. A retroviral envelope protein having a deletion of some, most, or all of the cytoplasmic domain is useful, because such manipulation results in hyperfusogenic activity for human cells. Particular modifications are introduced, in some embodiments, into viral membrane glycoproteins to enhance their function to induce cell membrane fusion. For example, truncation of the cytoplasmic domains of a number of retroviral and herpes virus glycoproteins has been shown to increase their fusion activity, sometimes with a simultaneous reduction in the efficiency with which they are incorporated into virions (Rein et al., (1994) J Virol 68(3): 1773-81).

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Construction of FusOn-H2

Initially, the HSV genome region comprising the ICP10 left-flanking region (equivalent to nucleotide number of HSV-2 genome 85994-86999) was amplified with the following exemplary pair of primers: 5′-TTGGTCTTCACCTACCGACA (SEQ ID NO: 1); and 3′-GACGCGATGAACGGAAAC (SEQ ID NO: 2). The RR domain and the right-flank region (equivalent to the nucleotide sequence number of HSV-2 genome 88228-89347) were amplified with the following exemplary pair of primers: 5′-ACACGCCCTATCATCTGAGG (SEQ ID NO: 13); and 5′-AACATGATGAAGGGGCTTCC (SEQ ID NO: 14). These two PCR products were cloned into pNeb 193 through EcoRI-NotI-XbaI ligation to generate pNeb-ICP10-deltaPK. Then, the DNA sequence containing the CMV promoter-EGFP gene was PCR amplified from pSZ-EGFP with the following exemplary pair of primers: 5′-ATGGTGAGCAAGGGCGAG (SEQ ID NO: 3); and 3′-CTTGTACAGCTCGTCCATGC (SEQ ID NO: 4). The PCR-amplified DNA was then cloned into the deleted PK locus of pNeb-ICP10-deltaPK through BglII and NotI ligation to generate pNeb-PKF-2. During the design of PCR amplification strategies, the primers were designed such that the EGFP gene was fused in frame with the remaining RR domain of the ICP10 gene so that the new protein product of this fusion gene comprises the intact functional EGFP, which would facilitate the selection of the recombinant virus in the following experimental steps.

The modified ICP10 gene was inserted into the virus through homologous recombination by co-transfecting the pNeb-PKF-2 plasmid DNA with purified HSV-2 genomic DNA (strain 186) into Vero cells by lipofectamine. The recombinant virus was screened and identified by selecting GFP-positive virus plaques. During the screening process, it was noticed that all of the GFP-positive plaques showed clear syncytial formation of the infected cells, indicating that this modified virus induces widespread cell membrane fusion, in specific embodiments of the invention. A total of 6 plaques were picked. One of them, referred to as FusOn-H2, was selected for further characterization and for all of the subsequent experiments.

### Example 2

To systemically mutate the majority of the neutralizing epitopes on both gB and gD, as well as to enhance the complement antagonizing capability of the gC, FusOn-H2 was subjected to a series of selection in the presence of anti-HSV-2 sera. FusOn-H2 was initially subjected to the selection in cell culture in the presence of a mixture of serum collected from 5 rats that had been vaccinated with HSV and had high anti-HSV antibodies in blood. One of the main reasons to start this selection with rat sera is because they contain both natural immunoglobulins and mannan-binding lectin (MBL) that can activate complement against HSV, while mouse and human sera only contain one of these two activation mechanisms (MBL for mouse and natural immunoglobulins for human, respectively) (Wakimoto et al., 2002). After 7 consecutive rounds of selection under this rat sera mixture, one human serum that contains a high level of anti-HSV-2 antibodies was added into the rat sera and continued the selection.

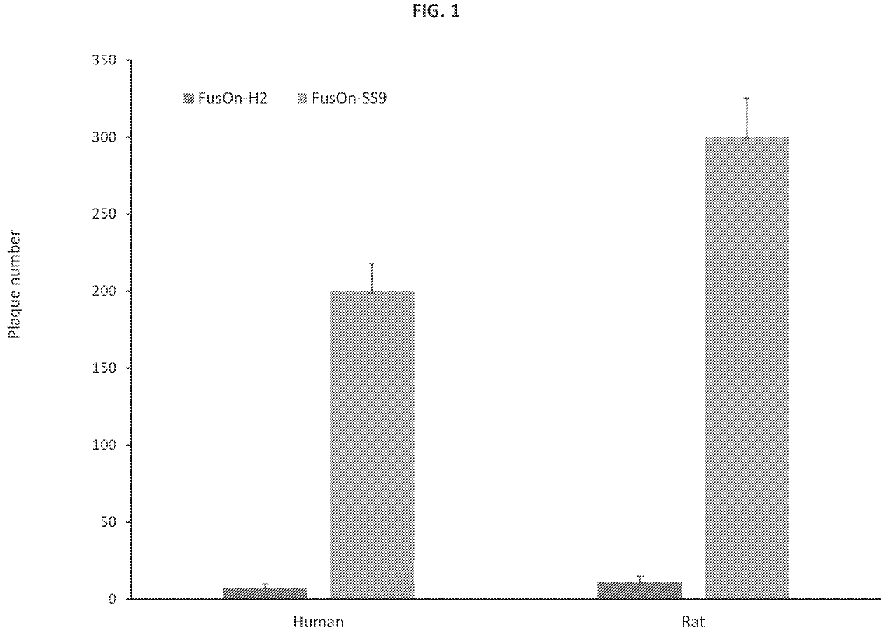

The virus infectivity in the presence of anti-HSV sera was monitored regularly during the selection process and the results indicated the continuous improvement over the unselected FusOn-H2 on the resistance to the neutralizing antibodies. One of the testing results is shown in FIG. 1, which was conducted after the virus underwent a total of 9 rounds of selection (i.e., 7 rounds of selection with the rat sera and 2 rounds of selection with a mixture of rat sera and human serum). The result showed that the trained virus (designated FusOn-S S9) was more than 28.5- and 27.2-fold more resistant to the neutralizing effect of human and rat anti-HSV-2 sera, respectively.

To enable FusOn-H2 with the ability to escape the host's clearance by the mononuclear phagocyte system (MPS) in the blood during systemic delivery, the possibility of genetically engrafting CD47, a “don't eat me” signal molecule, to the membrane envelope of the virus was explored. Macrophages are the major phagocytes that are responsible for rapid clearance of HSV particles and it has been reported that depletion of macrophages can significantly improve the therapeutic effect of oncolytic HSV (Fulci et al., 2007). The phagocytic activity of macrophages is controlled by both positive and negative regulation mechanisms. Interactions between CD47 and its receptor, SIRPa, provide a strong negative regulation signal (“don't eat me signal”) to macrophages (Kinchen and Ravichandran, 2008).

HSV encodes several glycoproteins that are assembled on the surface of viral envelope. They include glycoprotein C (gC), gB, gD, gH and gL. Each of them can serve as a candidate molecule for incorporating the extracellular domain (ECD) of murine CD47 (mCD47) so that it may be engrafted to the surface of the virus envelope. Glycoprotein C (gC) was chosen here, as unlike other glycoproteins mentioned, it is not essential for virus infectivity. As such, modifying it for incorporating mCD47 would not run the risk of altering the natural tropism of the oncolytic virus. The ECD of mCD47 (aa 19-141) (SEQ ID NO: 16) (shown in the table below) was initially inserted into the N-terminus of gC to create the chimeric form of gC (cgC), and its expression is driven by CMV IE promoter.

| | |
|---|---|
| Full length CD47 | QLLFNKTKSV EFTFCNDTVV IPCFVTNMEA |
| (SEQ ID NO: 16) | QNTTEVYVKW KFKGRDIYTF DGALNKSTVP |
| | TDFSSAKIEV SQLLKGDASL KMDKSDAVSH |
| | TGNYTCEVTE LTREGETIIE LKYRVVSWFS |
| | PNE |
| Amino acids | MWPLVAALLL GSACCGSAQL LFNKTKSVEF |
| 19-141 of CD47 | TFCNDTVVIP CFVTNMEAQN TTEVYVKWKF |
| (SEQ ID NO: 21) | KGRDIYTFDG ALNKSTVPTD FSSAKIEVSQ |
| | LLKGDASLKM DKSDAVSHTG NYTCEVTELT |
| | REGETIIELK YRVVSWFSPN ENILIVIFPI |
| | FAILLFWGQF GIKTLKYRSG GMDEKTIALL |
| | VAGLVITVIV IVGAILFVPG EYSLKNATGL |
| | GLIVTSTGIL ILLHYYVFST AIGLTSFVIA |
| | ILVIQVIAYI LAVVGLSLCI AACIPMHGPL |
| | LISGLSILAL AQLLGLVYMK FVASNQKTIQ |
| | PPRKAVEEPL NAFKESKGMM NDE |

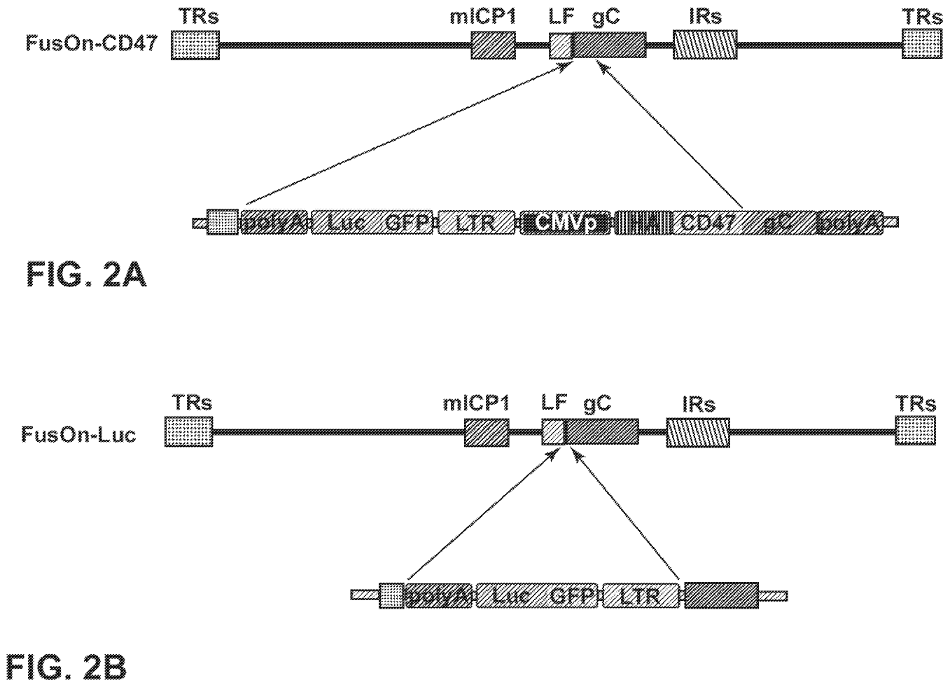

A HA (hemagglutinin) tag was included in cgC to allow the chimeric molecule to be conveniently detected. For the purpose of easiness in identifying the recombinant virus and for in vivo imaging, cgC was linked to another gene cassette that contains the EGFP-luciferase gene. These two gene cassettes were inserted together into the backbone of FusOn-H3, which was derived from a HSV-2 based oncolytic virus (FusOn-H2) by deleting the GFP gene from the virus. FusOn-H2 was constructed by deleting and replacing the N-terminal region of the ICP10 gene with GFP to render it the ability to selectively replicate in and kill tumor cells. The recombinant virus was identified by picking up GFP positive plaques. Each individually picked virus was enriched to homogenous GFP positivity through multiple rounds of plaque purification. The newly generated virus is designated FusOn-CD47-Luc (FIG. 2A). A control virus was also constructed, in which only the EGFP-Luc gene cassette alone was inserted into the backbone of FusOn-H3, FusOn-Luc (FIG. 2B). All the selected viruses maintain the fusogenic property of the parental FusOn-H2.

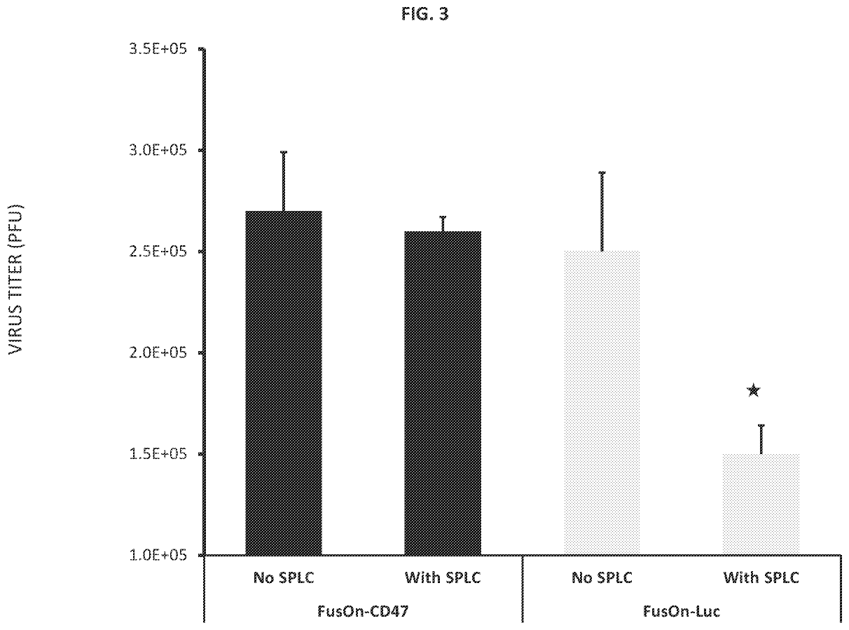

An interesting recent study finds that coating nanoparticles with a CD47 mimetic peptide can help them escape phagocytic clearance by MPS (Rodriguez et al., 2013). The possibility of genetically engrafting the extracellular domain of CD47 molecule to the membrane envelop of an FusOn-H2 was explored, in order to enable it to escape from MPS for systemic delivery. The virus construction strategy is shown in FIG. 2. In vitro assays show that coating the viral particles with CD47 extracellular domain through the recombined gC makes the virus more resistant to the phagocytosis (FIG. 3). When tested in vivo in a murine colon cancer model, the results showed that FusOn-CD47 is 15-fold more effectively than FusOn-Luc to be delivered to the tumor site by the systemic route. Moreover, the data also showed that FusOn-CD47 could persist significantly longer than FusOn-Luc in the tumor site after systemic delivery.

The expression of the transgene by either flow cytometry or western blot analysis was initially examined. For flow cytometry analysis, 293 cells were infected with 1 pfu/cell of either FusOn-CD47-Luc or FusOn-Luc. Twenty-four hours later, cells were labeled with either a PE conjugated anti-mCD47 antibody or a rabbit anti-HA tag antibody. A goat-anti-rabbit antibody conjugated with FITC was added for HA tag detection. Both CD47 and HA tag labelled cells were subjected to flow cytometry analysis. The results in FIG. 2A show that both anti-mCD47 and anti-HA tag antibodies were able to readily detect the cells that had been infected with FusOn-CD47-Luc, but not cells infected with FusOn-Luc. For western blot analysis, 293 cells were either similarly infected with these two viruses or transfected with plasmids that express cgC or wild type gC that does not contain the HA tag. Cell lysates were prepared 24 h later and were subjected to western blot analysis. The result in FIG. 2B shows that cgC is abundantly expressed by FusOn-CD47-Luc but not FusOn-Luc.

Next, quantify of Luc gene expression from these two viruses was compared. Vero, CT26 and 4T1 cells were infected with either FusOn-CD47-Luc or FusOn-Luc at 1 pfu/cell. Cells were harvested 24 h later for measurement of luciferase activity. The result in FIG. 3A showed a near identical level of luciferase activity from cells infected with these two viruses. Another experiment was then conducted to determine, in the in vitro setting, if engrafting an oncolytic HSV with the ECD of mCD47 allows the virus to evade the engulfment and clearance by phagocytes. Mouse splenocytes were used as the source of fresh phagocytes as spleen is the largest unit of the mononuclear phagocyte system. Vero cells were infected with FusOn-CD47-Luc or FusOn-Luc with or without the presence of mouse splenocytes. Cells were collected 48 h later for quantitative measurement of virus yield by plaque assay. The results in FIG. 3B showed that, in the wells without splenocytes, both FusOn-CD47-Luc and FusOn-Luc replicated well and the virus yield was similar in these two wells. However, in the wells with splenocytes, FusOn-Luc yield was significantly reduced while the replication of FusOn-CD47-Luc was only marginally affected. These results indicate that the presence of mCD47 ECD on viral particles enable the virus with the ability to resist the impact from macrophages and possibly some other immune cells during virus infection.

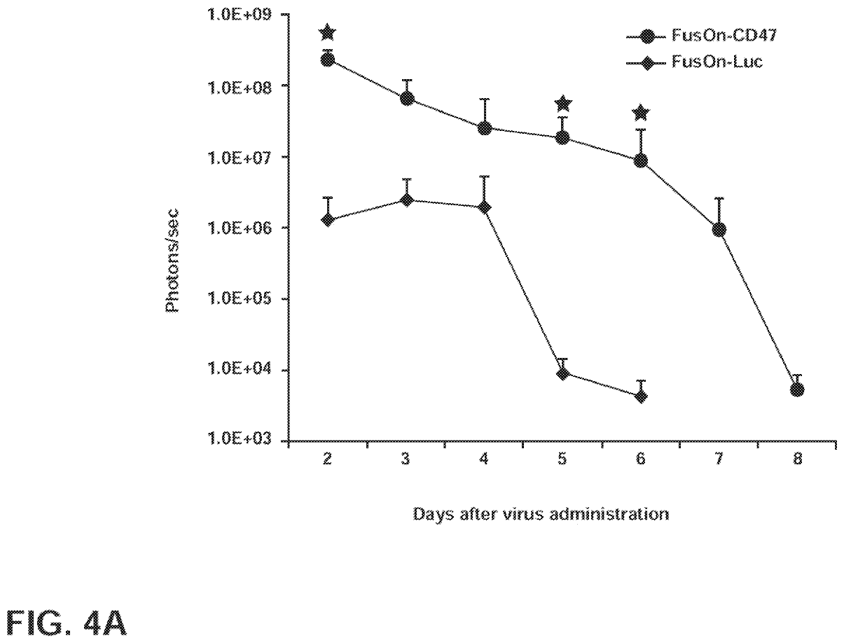

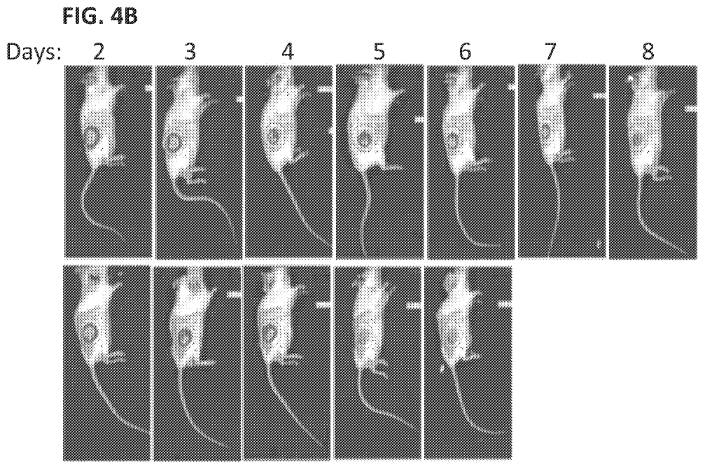

To determine the ability of the incorporated mCD47 ECD in enabling the virus for systemic delivery, CD26 murine colon cancer cells were implanted to the right flank of immunocompetent Balb/c mice. Once tumors reached the approximate size of 8 mm in diameter, we systemically injected 2×106 pfu of either FusOn-CD47-Luc or FusOn-Luc to each mouse. Mice were monitored for luciferase activity by IVIS Spectrum System starting on day 2 and then on a daily basis until total disappearance of the signal. The results in FIG. 4 show that, at day 2 after virus administration, the imaging signal from FusOn-CD47-Luc was approximately one and a half log stronger than that from FusOn-Luc. This indicates that the former was more efficiently delivered to the tumor site than later by the systemic route. Moreover, FusOn-CD47-Luc seems to stay in the tumor tissues substantially longer than FusOn-Luc. By day 5, the imaging signal was barely detectable in tumors from mice receiving FusOn-Luc, while the signal in tumors from FusOn-CD47-Luc remained detectable until day 7. Nevertheless, neither virus showed any significant amplification in tumor tissues because this HSV-2 based oncolytic virus grows poorly in CT26 tumor cells. Interestingly, in one occasion when a few mice were imaged at day 1 after virus delivery, significant imaging signals were detected transiently in the liver. These signals completely disappeared by day 2.

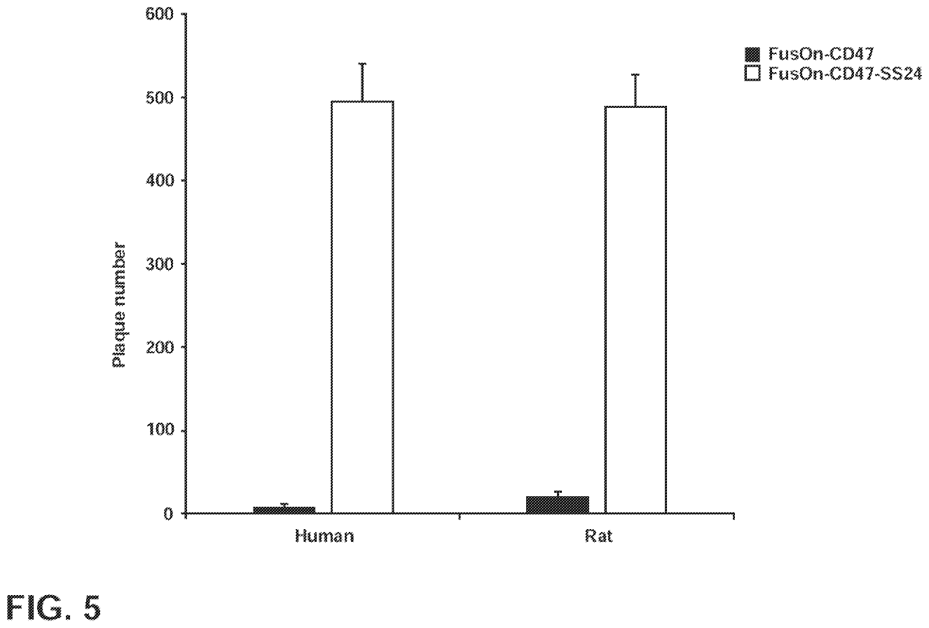

To determine if FusOn-CD47-Luc is also more superior than FusOn-Luc for systemic delivery in other tumor models, the above experiment was repeated, but in mice bearing tumors at the right flank established from implantation of 4T1 murine mammary tumor cells, in which FusOn-H2 was found to be able to grow moderately. Additionally, 4T1 tumor cells secrete macrophage colony stimulating factor (M-CSF) and granulocyte colony stimulating factor (G-CSF), which can enhance macrophage infiltration and phagocytosis. This would allow the CD47-mediated evading strategy to be more vigorously tested. The IVIS imaging results in FIG. 5A indeed showed that the signal in 4T1 tumor was in general lower than those detected in CT26 tumor (as shown in FIG. 4). Nevertheless, the results again showed that FusOn-CD47-Luc could be more efficiently delivered to local tumors than FusOn-Luc by the systemic route. By day 2, difference of the image signal strength between these two viruses was about one and half a log. The biggest difference was recorded on day 4, when the image signal from FusOn-Luc was reduced to nearly background level while it reached the highest for FusOn-CD47-Luc. These data again showed that CD47 modification allows the virus to be more efficiently delivered by the systemic route as well as persisted in tumor tissues longer than the control virus once it had reached there.

To generate a product of FusOn-H2 that can withstand all the three major limiting factors in the bloodstream—the neutralizing antibodies, the complements, and the phagocytes, we subjected FusOn-CD47 to the anti-HSV-2 sera selection as described above. It was subjected to 7 consecutive section with anti-HSV rat sera and then 23 consecutive passage in the presence of rat sera plus one human serum with an extremely high level of anti-HSV-2 antibodies as described above Again, several tests were conducted during the selections to determine if the virus was gaining the ability to resist the neutralizing effect of the immune sera One of the results from such tests is shown in FIG. 5, which was conducted after FusOn-CD47 was first subjected to 7 rounds of section with the rat sera and 17 rounds of section with the rat sera plus the human serum. The results clearly show that, similar to FusOn-SS9, the trained FusOn-CD47 (designated FusOn-CD47-SS24) gained substantial ability in preserving the infectivity in the presence of immune anti-HSV-2 sera.

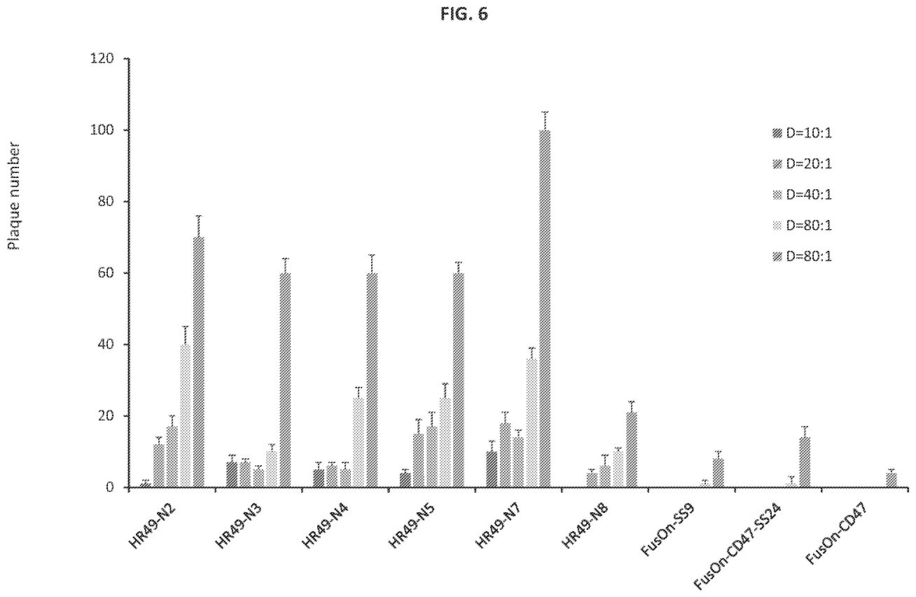

To ensure more profound resistance to the neutralizing antibodies in humans, we subjected the virus to three more rounds of section with a mixture of HSV-2 positive sera obtained from 12 individuals, 4 mixed together a time. The rounds of selection are: 8 for batch 1, 6 for batch 2, and 4 for batch 3. After these extensive selections, the obtained viruses were plaque purified and 6 plaque picks were expanded for further analysis. They are designated HR49-N2-5, HR49-N7 and HR49-N8. First, they were tested on their ability to resist the neutralization from a mixture of eight of the human sera used in the selection, which is the most stringent neutralization test that we have conducted. Both FusOn-SS9 and FusOn-CD47-SS24 were included in this experiment for comparison. The anti-HSV sera mixture was used at different dilutions to incubate with the indicated viruses, after which the virus infectivity was determined. The results in FIG. 6 show that, under this stringent neutralizing condition (containing a mixture of antiviral sera from multiple individuals), the selected virus can still produce a significant number of plaques while the unselected virus did not produce any plaque until the sera mixture was diluted by 80-fold. The data also showed the subsequent three more rounds of selection in the human sera mixtures have further enhanced the ability of the selected virus in their ability to resist the neutralizing effect. Also, the data showed that some of the picked viruses have a better capability than others in resisting the neutralization by the mixed human antiviral sera.

### Example 3

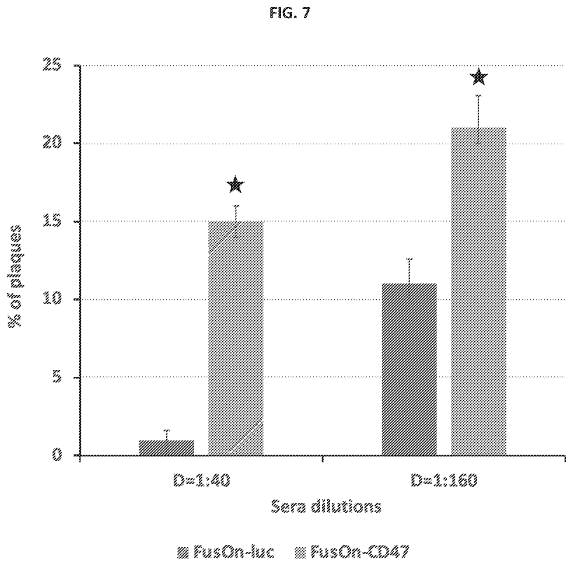

The ability of the oncolytic virus FusOn-H2, with and without insertion of an extracellular domain of CD47 into the N-terminus of gC, to escape neutralization by anti-HSV antibodies was evaluated. In this in vitro experiment, 500 plaque-forming units (pfu) of either FusOn-CD47 or FusOn-Luc were mixed with or without the presence of the diluted anti-HSV-2 sera (at a dilution of 1:40 or 1:160) and incubated at 37° C. before they were applied to Vero cell monolayers for assaying the infectivity and for quantification of plaque formation. The results in FIG. 7 showed that, at 1:40 dilution, the sera could neutralize the virus infectivity of FusOn-Luc almost completely. Even at 1:160 dilution, the sera could still significantly reduce the infection (and hence the number of plaque formation) of FusOn-Luc. In contrast, FusOn-CD47 was much less neutralized in the presence of the same diluted anti-HSV sera. In the presence of 1:40 diluted sera, FusOn-CD47 produced a significant number of plaques (approx.imately 15% of the well where no anti-HSV sera were added) while FusOn-Luc barely showed any viral plaque at all.

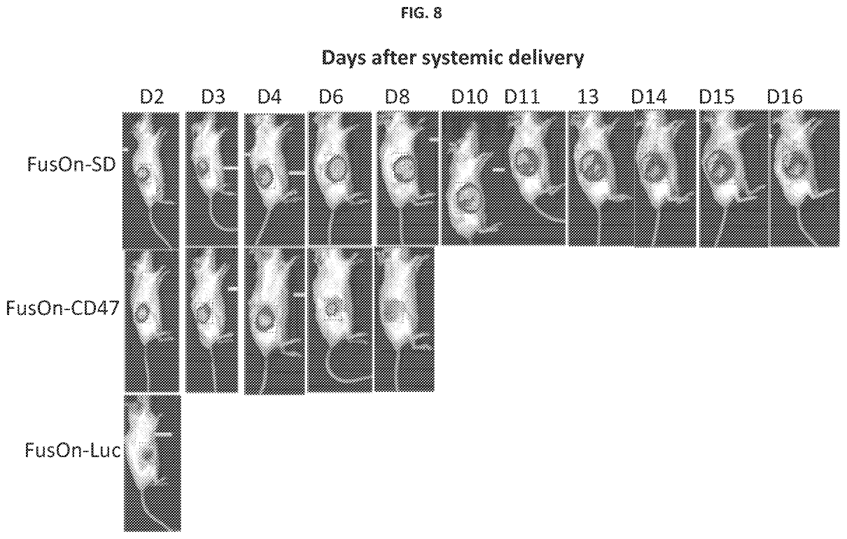

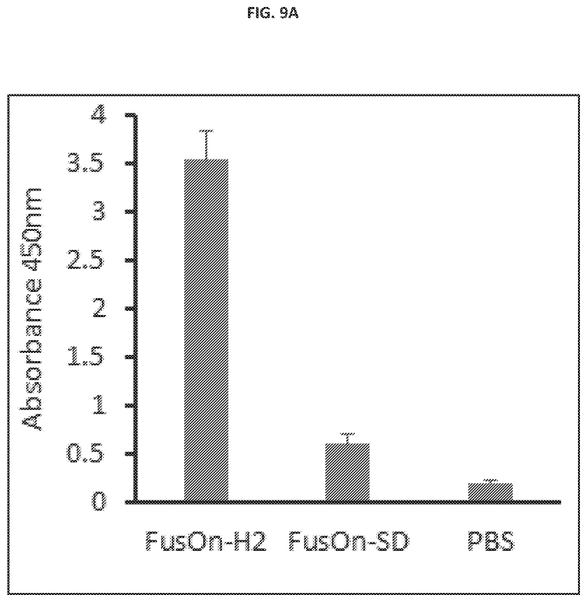

In vivo delivery by the systemic route was evaluated for FusOn-CD47, FusOn-Luc, and FusOn-SD in vaccinated Balb/c mice with HSV-2 and implanted with CT26 murine colon cancer cells in their right flank. 1×10⁷ of either FusOn-CD47, FusOn-Luc, or FusOn-SD was systemically delivered into the mice in different groups by injection via the tail vein. The mice were imaged on the days indicated in FIG. 8 with an IVIS imager. The results showed that FusOn-Luc was unable to be delivered to tumors by the systemic route in the presence of anti-HSV immunity. In contrast, FusOn-CD47 was readily detected in the tumor tissue by the same delivery route and in the presence of anti-HSV immunity. Moreover, FusOn-CD47 persisted in the tumor tissues for almost eight days after reaching the tumor site despite the presence of antiviral immunity. CD47 unexpectedly assisted the oncolytic virus in escaping the neutralization effect by anti-HSV antibodies, contributing to the overall ability of FusOn-SD in escaping neutralizing antibodies for systemic delivery. Accordingly, FusOn-SD behaved the best in systemic delivery to the tumor site. FusOn-SD is thus useful in patients having HSV-1 and/or HSV-2 antibodies, such as those vaccinated against HSV-1 and/or HSV-2 or having HSV-1 and/or HSV-2.

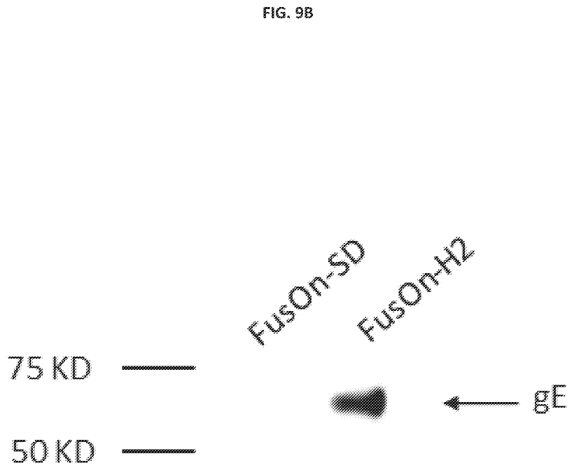

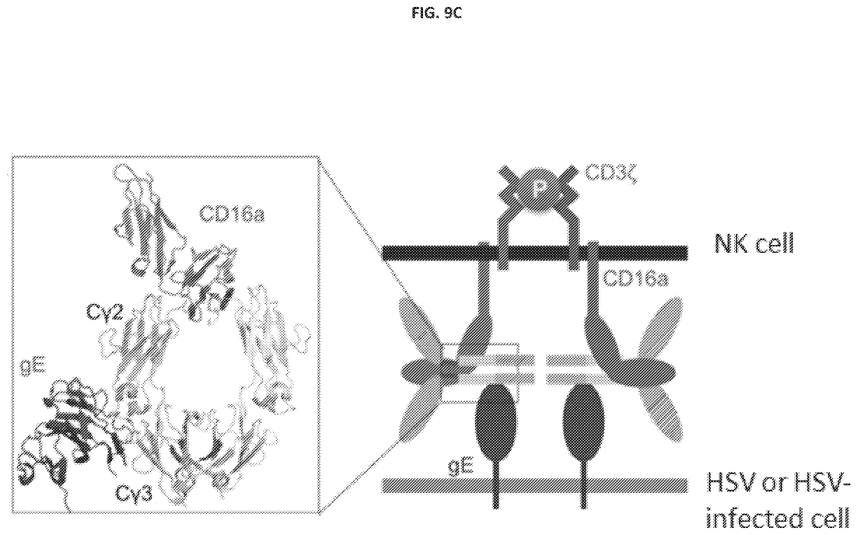

It was unexpectedly found that gE is absent from FusOn-SD viral particles. This is shown by FIGS. 9A to 9C.

FIG. 9A is a bar chart showing the absorbance of FusOn-H2, FusOn-SD, and PBS (negative control) after treatment with mouse anti-HSV-2 gE (1:1000 dilution) and HPR-conjugated rabbit anti-mouse IgG (1:10,000 dilution). More specifically, in FIG. 9A, 1×10⁶ pfu of viruses (the parental FusOn-H2 and FusOn-SD) were coated to 96-well plate for ELISA assay. Wells coated with PBS served as the negative control. The first antibody used was mouse anti-HSV-2 gE (1:1000 dilution) and the second antibody was HPR-conjugated rabbit anti-mouse IgG (1:10,000 dilution). A significant reading was detected in the FusOn-H2, but not in FusOn-SD, indicating that gE was present in FusOn-H2 viral particles, but not in FusOn-SD.

FIG. 9B is a western blot showing detection of gE for FusOn-SD and FusOn-H2. Proteins were subtracted from 1×10⁶ pfu of viruses (FusOn-H2 and FusOn-SD) and were run on an acrylamide gel for western blot analysis. The same antibodies of mouse anti-HSV-2 gE and HPR conjugated rabbit anti-mouse IgG used with regard to FIG. 9A were used for the western blot. The result showed a clear band of gE for FusOn-H2, while it was absent from the lane loaded with proteins from FusOn-SD. This result confirms the finding in FIG. 9A.

FIG. 9C is a schematic illustration on the mechanism of NK cell recognition of HSV or HSV infected cells via gE. The drawing shows that gE binds to IgG (either virus specific or non virus specific). The CD16a, which is one the most important NK cell activation receptors, then binds to the Fc region of the IgG, leading to the activation of NK cells and the clearance of either viral particles or the virus infected cells. This leads to reduced oncolytic virus delivery as well as replication, hence, minimizing the therapeutic effect of virotherapy. Because FusOn-SD is not recognized by this mechanism, NK cells do not clear them out and the therapeutic efficacy of FusOn-SD persists.

### REFERENCES

Bender, F. C., Samanta, M., Heldwein, E. E., de Leon, M. P., Bilman, E., Lou, H., Whitbeck, J. C., Eisenberg, R. J. and Cohen, G. H., 2007. Antigenic and mutational analyses of herpes simplex virus glycoprotein B reveal four functional regions. J Virol 81, 3827-41.
Cairns, T. M., Huang, Z. Y., Gallagher, J. R., Lin, Y., Lou, H., Whitbeck, J. C., Wald, A., Cohen, G. H. and Eisenberg, R. J., 2015. Patient-Specific Neutralizing Antibody Responses to Herpes Simplex Virus Are Attributed to Epitopes on gD, gB, or Both and Can Be Type Specific. J Virol 89, 9213-31.
Cairns, T. M., Huang, Z. Y., Whitbeck, J. C., Ponce de Leon, M., Lou, H., Wald, A., Krummenacher, C., Eisenberg, R. J. and Cohen, G. H., 2014. Dissection of the antibody response against herpes simplex virus glycoproteins in naturally infected humans. J Virol 88, 12612-22.
Eing, B. R., Kuhn, J. E. and Braun, R. W., 1989. Neutralizing activity of antibodies against the major herpes simplex virus type 1 glycoproteins. J Med Virol 27, 59-65.
Ellermann-Eriksen, S., 2005. Macrophages and cytokines in the early defence against herpes simplex virus. Virol J 2, 59.
Fu, X., Tao, L., Li, M., Fisher, W. E. and Zhang, X., 2006. Effective treatment of pancreatic cancer xenografts with a conditionally replicating virus derived from type 2 herpes simplex virus. Clin Cancer Res 12, 3152-3157.
Fu, X. and Zhang, X., 2001. Delivery of herpes simplex virus vectors through liposome formulation. Mol Ther 4, 447-53.
Fulci, G., Dmitrieva, N., Gianni, D., Fontana, E. J., Pan, X., Lu, Y., Kaufman, C. S., Kaur, B., Lawler, S. E., Lee, R. J., Marsh, C. B., Brat, D. J., van Rooij en, N., Stemmer-Rachamimov, A. O., Hochberg, F. H., Weissleder, R., Martuza, R. L. and Chiocca, E. A., 2007. Depletion of peripheral macrophages and brain microglia increases brain tumor titers of oncolytic viruses. Cancer Res 67, 9398-406.
Greig, S. L., 2016. Talimogene Laherparepvec: First Global Approval. Drugs 76, 147-54.
Huber, V. C., Lynch, J. M., Bucher, D. J., Le, J. and Metzger, D. W., 2001. Fc Receptor-Mediated Phagocytosis Makes a Significant Contribution to Clearance of Influenza Virus Infections. The Journal of Immunology 166, 7381-7388.
Hume, D. A., 2006. The mononuclear phagocyte system. Curr Opin Immunol 18, 49-53.
Kinchen, J. M. and Ravichandran, K. S., 2008. Phagocytic signaling: you can touch, but you can't eat. Curr Biol 18, R521-4.
Macedo, N., Miller, D. M., Haq, R. and Kaufman, H. L., 2020. Clinical landscape of oncolytic virus research in 2020. Journal for ImmunoTherapy of Cancer 8, e001486.
Nakamori, M., Fu, X., Pettaway, C. A. and Zhang, X., 2004. Potent antitumor activity after systemic delivery of a doubly fusogenic oncolytic herpes simplex virus against metastatic prostate cancer. Prostate 60, 53-60.
Rodriguez, P. L., Harada, T., Christian, D. A., Pantano, D. A., Tsai, R. K. and Discher, D. E., 2013. Minimal “Self” peptides that inhibit phagocytic clearance and enhance delivery of nanoparticles. Science 339, 971-5.
Silke Heilingloh, C., Lull, C., Kleiser, E., Alt, M., Schipper, L., Witzke, O., Trilling, M., Eis-Hubinger, A. M., Dittmer, U. and Krawczyk, A., 2020. Herpes Simplex Virus Type 2 Is More Difficult to Neutralize by Antibodies Than Herpes Simplex Virus Type 1. Vaccines (Basel) 8.
Tay, M. Z., Wiehe, K. and Pollara, J., 2019. Antibody-Dependent Cellular Phagocytosis in Antiviral Immune Responses. Frontiers in immunology 10.
Tuzmen, C., Cairns, T. M., Atanasiu, D., Lou, H., Saw, W. T., Hall, B. L., Cohen, J. B., Cohen, G. H. and Glorioso, J. C., 2020. Point Mutations in Retargeted gD Eliminate the Sensitivity of EGFR/EGFRvIII-Targeted HSV to Key Neutralizing Antibodies. Molecular Therapy—Methods & Clinical Development 16, 145-154.
Van Strijp, J. A., Van Kessel, K. P., van der Tol, M. E., Fluit, A. C., Snippe, H. and Verhoef, J., 1989. Phagocytosis of herpes simplex virus by human granulocytes and monocytes. Arch Virol 104, 287-98.
Wakimoto, H., Ikeda, K., Abe, T., Ichikawa, T., Hochberg, F. H., Ezekowitz, R. A., Pasternack, M. S. and Chiocca, E. A., 2002. The complement response against an oncolytic virus is species-specific in its activation pathways. Mol Ther 5, 275-82.
Alvarez-Breckenridge, C. A., Yu, J., Price, R., Wei, M., Wang, Y., Nowicki, M. O., Ha, Y. P., Bergin, S., Hwang, C., Fernandez, S. A., Kaur, B., Caligiuri, M. A. and Chiocca, E. A., 2012a. The histone deacetylase inhibitor valproic acid lessens NK cell action against oncolytic virus-infected glioblastoma cells by inhibition of STATS/T-BET signaling and generation of gamma interferon. J Virol 86, 4566-77 (“Alvarez-Breckenridge et al., 2012a”).
Alvarez-Breckenridge, C. A., Yu, J., Price, R., Wojton, J., Pradarelli, J., Mao, H., Wei, M., Wang, Y., He, S., Hardcastle, J., Fernandez, S. A., Kaur, B., Lawler, S. E., Vivier, E., Mandelboim, O., Moretta, A., Caligiuri, M. A. and Chiocca, E. A., 2012b. NK cells impede glioblastoma virotherapy through NKp30 and NKp46 natural cytotoxicity receptors. Nat Med 18, 1827-34 (“Alvarez-Breckenridge et al., 2012b”).
Bender, F. C., Samanta, M., Heldwein, E. E., de Leon, M. P., Bilman, E., Lou, H., Whitbeck, J. C., Eisenberg, R. J. and Cohen, G. H., 2007. Antigenic and mutational analyses of herpes simplex virus glycoprotein B reveal four functional regions. J Virol 81, 3827-41.
Cairns, T. M., Huang, Z. Y., Gallagher, J. R., Lin, Y., Lou, H., Whitbeck, J. C., Wald, A., Cohen, G. H. and Eisenberg, R. J., 2015. Patient-Specific Neutralizing Antibody Responses to Herpes Simplex Virus Are Attributed to Epitopes on gD, gB, or Both and Can Be Type Specific. J Virol 89, 9213-31.
Cairns, T. M., Huang, Z. Y., Whitbeck, J. C., Ponce de Leon, M., Lou, H., Wald, A., Krummenacher, C., Eisenberg, R. J. and Cohen, G. H., 2014. Dissection of the antibody response against herpes simplex virus glycoproteins in naturally infected humans. J Virol 88, 12612-22.
Dai, H.-S. and Caligiuri, M. A., 2018. Molecular Basis for the Recognition of Herpes Simplex Virus Type 1 Infection by Human Natural Killer Cells. Frontiers in immunology 9, 183-183.
Dubin, G., Basu, S., Mallory, D. L., Basu, M., Tal-Singer, R. and Friedman, H. M., 1994. Characterization of domains of herpes simplex virus type 1 glycoprotein E involved in Fc binding activity for immunoglobulin G aggregates. J Virol 68, 2478-85.
Eing, B. R., Kühn, J. E. and Braun, R. W., 1989. Neutralizing activity of antibodies against the major herpes simplex virus type 1 glycoproteins. J Med Virol 27, 59-65.
Ellermann-Eriksen, S., 2005. Macrophages and cytokines in the early defence against herpes simplex virus. Virol J 2, 59.
Fu, X., Tao, L., Li, M., Fisher, W. E. and Zhang, X., 2006. Effective treatment of pancreatic cancer xenografts with a conditionally replicating virus derived from type 2 herpes simplex virus. Clin Cancer Res 12, 3152-3157.
Fu, X. and Zhang, X., 2001. Delivery of herpes simplex virus vectors through liposome formulation. Mol Ther 4, 447-53.
Fulci, G., Dmitrieva, N., Gianni, D., Fontana, E. J., Pan, X., Lu, Y., Kaufman, C. S., Kaur, B., Lawler, S. E., Lee, R. J., Marsh, C. B., Brat, D. J., van Rooij en, N., Stemmer-Rachamimov, A. O., Hochberg, F. H., Weissleder, R., Martuza, R. L. and Chiocca, E. A., 2007. Depletion of peripheral macrophages and brain microglia increases brain tumor titers of oncolytic viruses. Cancer Res 67, 9398-406.
Greig, S. L., 2016. Talimogene Laherparepvec: First Global Approval. Drugs 76, 147-54.
Huber, V. C., Lynch, J. M., Bucher, D. J., Le, J. and Metzger, D. W., 2001. Fc Receptor-Mediated Phagocytosis Makes a Significant Contribution to Clearance of Influenza Virus Infections. The Journal of Immunology 166, 7381-7388.
Hume, D. A., 2006. The mononuclear phagocyte system. Curr Opin Immunol 18, 49-53.
Kinchen, J. M. and Ravichandran, K. S., 2008. Phagocytic signaling: you can touch, but you can't eat. Curr Biol 18, R521-4.
Macedo, N., Miller, D. M., Haq, R. and Kaufman, H. L., 2020. Clinical landscape of oncolytic virus research in 2020. Journal for ImmunoTherapy of Cancer 8, e001486.
Nakamori, M., Fu, X., Pettaway, C. A. and Zhang, X., 2004. Potent antitumor activity after systemic delivery of a doubly fusogenic oncolytic herpes simplex virus against metastatic prostate cancer. Prostate 60, 53-60.
Rodriguez, P. L., Harada, T., Christian, D. A., Pantano, D. A., Tsai, R. K. and Discher, D. E., 2013. Minimal “Self” peptides that inhibit phagocytic clearance and enhance delivery of nanoparticles. Science 339, 971-5.
Silke Heilingloh, C., Lull, C., Kleiser, E., Alt, M., Schipper, L., Witzke, O., Trilling, M., Eis-Hubinger, A. M., Dittmer, U. and Krawczyk, A., 2020. Herpes Simplex Virus Type 2 Is More Difficult to Neutralize by Antibodies Than Herpes Simplex Virus Type 1. Vaccines (Basel) 8.
Tay, M. Z., Wiehe, K. and Pollara, J., 2019. Antibody-Dependent Cellular Phagocytosis in Antiviral Immune Responses. Frontiers in immunology 10.
Tuzmen, C., Cairns, T. M., Atanasiu, D., Lou, H., Saw, W. T., Hall, B. L., Cohen, J. B., Cohen, G. H. and Glorioso, J. C., 2020. Point Mutations in Retargeted gD Eliminate the Sensitivity of EGFR/EGFRvIII-Targeted HSV to Key Neutralizing Antibodies. Molecular Therapy—Methods & Clinical Development 16, 145-154.
Van Strijp, J. A., Van Kessel, K. P., van der Tol, M. E., Fluit, A. C., Snippe, H. and Verhoef, J., 1989. Phagocytosis of herpes simplex virus by human granulocytes and monocytes. Arch Virol 104, 287-98.
Wakimoto, H., Ikeda, K., Abe, T., Ichikawa, T., Hochberg, F. H., Ezekowitz, R. A., Pasternack, M. S. and Chiocca, E. A., 2002. The complement response against an oncolytic virus is species-specific in its activation pathways. Mol Ther 5, 275-82.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

1. A composition comprising an oncolytic Herpes Simplex Virus Type 1 (HSV-1) or Herpes Simplex Virus Type 2 (HSV-2), wherein the oncolytic HSV1 or HSV-2 is prepared by passaging at least twice an oncolytic HSV1 or HSV-2 with immune sera having elevated levels of anti-HSV antibodies.

2. The composition of claim 1, wherein the oncolytic HSV1 or HSV-2 has a membrane envelope comprising glycoproteins, wherein at least one glycoprotein comprises an extracellular CD47 domain inserted into the N-terminus of a glycoprotein.

3. The composition of claim 2, wherein the glycoprotein is selected from glycoprotein C, glycoprotein B, glycoprotein D, glycoprotein H, and glycoprotein L.

4. The composition of claim 3, wherein the glycoprotein is glycoprotein C.

5. The composition of claim 1, wherein the passaging in the immune sera mutates neutralizing epitopes on glycoprotein B and glycoprotein D of the oncolytic HSV-1 or HSV-2.

6. The composition of claim 2, wherein the extracellular CD47 domain comprises amino acids 19-141 of CD47.

7. The composition of claim 2, wherein the oncolytic HSV1 or HSV-2 having an extracellular CD47 domain is free or substantially free of gE.

8. A composition comprising an oncolytic Herpes Simplex Virus Type 1 or Herpes Simplex Virus Type 2 (HSV-2), wherein the oncolytic HSV1 or HSV-2 is prepared by passaging at least twice an oncolytic HSV-1 or HSV-2 in a mixture of immune sera, wherein the mixture of immune sera is comprised of rat sera and human sera that has elevated levels of anti-HSV antibodies.

9. The composition of claim 1, wherein the composition comprises oncolytic HSV-2, which has been prepared by passaging seven times an oncolytic HSV-2 in the presence of rat sera having an elevated level of anti-HSV antibodies followed by passaging seventeen times in the presence of a mixture of rat sera and at least one human serum having an elevated level of anti-HSV antibodies.

10. The composition of claim 1, wherein the composition comprises oncolytic HSV-2, which has been prepared by passaging seven times an oncolytic HSV-2 in the presence of rat sera having an elevated level of anti-HSV antibodies, followed by passaging twenty-three times in the presence of a mixture of rat sera and at least one human serum having an elevated level of anti-HSV antibodies.

11. The composition of claim 1, wherein the oncolytic HSV-1 or oncolytic HSV-2 comprises a modified ICP10 coding region lacking nucleotides 1 to 1204 of an endogenous ICP10 coding region, wherein the oncolytic HSV-1 or HSV-2 comprises the modified ICP10 operably linked to an endogenous or a constitutive promoter and expresses a modified ICP10 polypeptide that lacks protein kinase (PK) activity but retains ribonucleotide reductase activity; and wherein the oncolytic HSV-1 or HSV-2 is capable of selectively killing cancer cells.

12. The composition of claim 1, wherein the composition comprises an oncolytic HSV-2, and the oncolytic HSV-2 subject to passaging is FusOn-H2 oncolytic virus.

13. The composition of claim 1, wherein the composition comprises an oncolytic HSV-2, and the oncolytic HSV-2 subject to passaging is FusOn-CD47 oncolytic virus.

14. A method of preparing a composition comprising an oncolytic Herpes Simplex Virus Type 1 (HSV-1) or Herpes Simplex Virus Type 2 (HSV-2) comprising passaging at least twice an oncolytic Herpes Simplex Virus Type 1 (HSV-1) or Herpes Simplex Virus Type 2 (HSV-2) with immune sera that has elevated levels of anti-HSV antibodies.

15. The method of claim 14, wherein the oncolytic HSV1 or HSV-2 has a membrane envelope comprising glycoproteins, wherein at least one glycoprotein comprises an extracellular CD47 domain inserted into the N-terminus of a glycoprotein.

16. The method of claim 15, wherein the glycoprotein is selected from glycoprotein C, glycoprotein B, glycoprotein D, glycoprotein H, and glycoprotein L.

17. The method of claim 16, wherein the glycoprotein is glycoprotein C.

18. The method of claim 14, wherein the passaging in the immune sera mutates neutralizing epitopes on glycoprotein B and glycoprotein D of the oncolytic HSV-1 or HSV-2.

19. The method of claim 15, wherein the extracellular domain comprises amino acids 19-141 of CD47.

20. The method of claim 14, wherein the immune sera comprises mammalian anti-HSV antibodies.

21. The method of claim 14, wherein the method comprises passaging the oncolytic HSV-2 at least two times in the presence of rat sera having an elevated level of anti-HSV antibodies followed by passaging at least once in the presence of a mixture of rat sera and at least one human serum having an elevated level of anti-HSV antibodies.

22. The method of claim 21, wherein the method comprises passaging seven times the oncolytic HSV-2 in the presence of rat sera having an elevated level of anti-HSV antibodies followed by passaging seventeen times in the presence of a mixture of rat sera and at least one human serum having an elevated level of anti-HSV antibodies.

23. The method of claim 21, wherein the method comprises passaging seven times the oncolytic HSV-2 in the presence of rat sera having an elevated level of anti-HSV antibodies followed by passaging twenty-three times in the presence of a mixture of rat sera and at least one human serum having an elevated level of anti-HSV antibodies.

24. The method of claim 14, wherein the composition comprises an oncolytic HSV-2, and the oncolytic HSV-2 subject to passaging is FusOn-H2 oncolytic virus.

25. A method of treating cancer in a patient in need thereof with an HSV-based oncolytic virotherapy comprising administering a composition of claim 1.

26. The method of claim 25, wherein the composition is systemically administered.

27. The method of claim 25 or 26, wherein the cancer is metastatic cancer.

28. A method of treating cancer in a patient in need thereof with an HSV-based oncolytic virotherapy comprising administering a composition prepared by the method of claim 15.

29. The method of claim 28, wherein the composition is systemically administered.

30. The method of claim 28, wherein the composition is administered by intratumoral injection or by intraperitoneal injection.

31. The method of claim 28, wherein the cancer is metastatic cancer.

32. The method of claim 25, wherein the patient has been vaccinated against HSV-1 and/or HSV-2, or has HSV-1 and/or HSV-2.

33. The method of claim 25, wherein the method further comprises treating the patient with a checkpoint blockage immunotherapy.

34. The method of claim 33, wherein the checkpoint blockage immunotherapy is selected from (a) administering to the patient a PD-L1 inhibitor, a PD-1 inhibitor, or a CTLA-4 inhibitor, or (b) treating the patient by an adoptive T-cell transfer.


SEQUENCE LISTING (21 sequences)

SEQ ID NO: 1 - DNA, 20 bp, Artificial Sequence
  Description of Artificial Sequencesynthetic amplification primer for left-flanking region of Herpes simplex virus-2 (HSV-2) ribonucleotide reductase large subunit (ICP10, RR1)
ttggtcttcacctaccgaca

SEQ ID NO: 2 - DNA, 18 bp, Artificial Sequence
  Description of Artificial Sequencesynthetic amplification primer for left-flanking region of Herpes simplex virus-2 (HSV-2) ribonucleotide reductase large subunit (ICP10, RR1)
caaaggcaagtagcgcag

SEQ ID NO: 3 - DNA, 18 bp, Artificial Sequence
  Description of Artificial Sequencesynthetic PCR amplification primer for constitutive immediate early cytomegalovirus (CMV) promoter and enhanced green fluorescent protein (EGFP)
atggtgagcaagggcgag

SEQ ID NO: 4 - DNA, 20 bp, Artificial Sequence
  Description of Artificial Sequencesynthetic PCR amplification primer for constitutive immediate early cytomegalovirus (CMV) promoter and enhanced green fluorescent protein (EGFP)
cgtacctgctcgacatgttc

SEQ ID NO: 5 - DNA, 120 bp, Artificial Sequence
  Description of Artificial SequenceC-type retrovirus gibbon ape leukemia virus (GALV) fusogenic membrane envelope glycoprotein (FMG) domain
ttaagcctggtaccgtaacaatccctcacccgttccaggtcggggatcaagtgcttgtcagacgccatcgacccagcagccttgagcctcggtggaaaggcccatacctggtgttgctga

SEQ ID NO: 6 - DNA, 2890 bp, Artificial Sequence
  Description of Artificial SequenceC-type retrovirus murine leukemia virus (MLV) fusogenic membrane envelope glycoprotein (FMG) domain
acttgtggtctcgctgttccttgggagggtctcctctgagtgattgactaccgtcagcgggggtctttcatttgggggctcgtccgggatcgggagacccctgcccagggaccaccgacccaccaccgggagctcacttacaggcccttcaagcagtacaacgagaggtctggaagccactggctgcggcctatcaggaccagcaagaccagccagtgataccacaccccttccgtgtcggcgacaccgtgtgggtacgccggcaccagactaagaacttggaacctcgttggaaaggaccctataccgtcctgctgaccacccccaccgctctcaaagtagacggcatcgctgcgtggatccacgccgctcacgtaaaggcggcgacaacccctccggccggaacagcatcaggaccgacatggaaggtccagcgttctcaaaaccccttaaagataagattaacccgtggggccccctgatagtcctggggatcttaataagggcaggagtatcagtacaacatgacagccctcaccaggtcttcaatgttacttggagagttaccaacttaatgacaggacaaacagctaacgctacctccctcctggggacaatgacagatgcctttcctatgctgtacttcgacttgtgcgatttaataggggacgattgggatgagactggacttgggtgtcgcactcccgggggaagaaaacgggcaagaacatttgacttctatgtttgccccgggcatactgtaccaacagggtgtggagggccgagagagggctactgtggcaaatggggctgtgagaccactggacaggcatactggaagccatcatcatcatgggacctaatttcccttaagcgaggaaacacccctcggaatcagggcccctgttatgattcctcagtggtctccagtggcatccagggtgccacaccggggggtcgatgcaatcccctagtcctagaattcactgacgcgggtaaaaaggccagctgggatggccccaaagtatggggactaagactgtaccaatccacagggatcgacccggtgacccggttctctttgacccgccaggtcctcaatatagggccccgcatccccattgggcctaatcccgtgatcactggccaactacccccctcccgacccgtgcagatcaggctccccaggcctcctcagactcctcctacaggcgcagcctctatggtccctgggactgccccaccgtctcaacaacctgggacgggagacaggctgctaaacctggtagatggagcataccaagcactcaacctcaccagtcctgacaaaacccaagagtgctggttgtgtctggtatcgggacccccctactacgaaggggttgccgtcctaggtacttactccaaccatacctctgccccagctaactgctccgcggcctcccaacacaagctgaccctgtccgaagtaaccggacagggactctgcgtaggagcagttcccaaaacccatcaggccctgtgtaataccacccaaaagacgagcgacgggtcctactatctggctgctcccgccgggaccatttgggcttgcaacaccgggctcactccctgcctatctactactgtactcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccg

SEQ ID NO: 7 - DNA, 2384 bp, Measles virus
  Measles virus fusion protein
tcgagggccaaggaacatacacacccaacagaacccagaccccggcccacggcgccgcgcccccaacccccgacaaccagagggagcccccaaccaatccgccggctcccccggtgcccacaggcagggacaccaacccccgaacagacccagcacccaaccatcgacaatccaagacgggggggcccccccaaaaaaaaggcccccaggggccgacagccagcaccgcgaggaagcccacccaccccacacacgaccacggcaaccaaaccagaacccagaccaccctgggtcaccagctccagacctcggtcatcaccccgcagaaaggaaaggcacaacccgcgaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccaccacacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgaggcaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagcttacgggaccccatatctgcggagatatctatccaggctttgagctatgcgcttggaggagacatcaataaggtgttagaaaagctcggatacagtggaggtgatttactgggcatcttagagagcagaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtccaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgttcccacaagtctcctcttcgtcatcaagcaaccaccgcacccagcatcaagcccacctgaaccagctaaattatctccggcttccctctggccgaacaatatcggtagttaatt

SEQ ID NO: 8 - DNA, 2535 bp, Human immunodeficiency virus
  HIV gp160
atgagagtgatggggatacagaggaattggccacaatggtggatatggggcaccttaggcttttggatgataataatttgtagggtggtggggaacttgaacttgtgggtcacagtctattatggggtacctgtgtggaaagaagcaaaaactactctattctgtgcatcagatgctaaagcatatgataaagaagtacataatgtctgggctacacatgcctgtgtacccacagaccccaacccacgagaaatagttttggaaaatgtaacagaaaattttaacatgtggaaaaatgacatggtggatcagatgcatgaggatataatcagtttatgggatcaaagcctaaaaccatgtgtaaagttgaccccactctgtgtcactttaaattgtacaaatgcacctgcctacaataatagcatgcatggagaaatgaaaaattgctctttcaatacaaccacagagataagagataggaaacagaaagcgtatgcacttttttataaacctgatgtagtgccacttaataggagagaagagaataatgggacaggagagtatatattaataaattgcaattcctcaaccataacacaagcctgtccaaaggtcacttttgacccaattcctatacattattgtgctccagctggttatgcgattctaaagtgtaataataagacattcaatgggacaggaccatgcaataatgtcagcacagtacaatgtacacatggaattatgccagtggtatcaactcaattactgttaaatggtagcctagcagaagaagagataataattagatctgaaaatctgacaaacaatatcaaaacaataatagtccaccttaataaatctgtagaaattgtgtgtacaagacccaacaataatacaagaaaaagtataaggataggaccaggacaaacattctatgcaacaggtgaaataataggaaacataagagaagcacattgtaacattagtaaaagtaactggaccagtactttagaacaggtaaagaaaaaattaaaagaacactacaataagacaatagaatttaacccaccctcaggaggggatctagaagttacaacacatagctttaattgtagaggagaatttttctattgcaatacaacaaaactgttttcaaacaacagtgattcaaacaacgaaaccatcacactcccatgcaagataaaacaaattataaacatgtggcagaaggtaggacgagcaatgtatgcccctcccattgaaggaaacataacatgtaaatcaaatatcacaggactactattgacacgtgatggaggaaagaatacaacaaatgagatattcagaccgggaggaggaaatatgaaggacaattggagaagtgaattatataaatataaagtggtagaaattgagccattgggagtagcacccactaaatcaaaaaggagagtggtggagagagaaaaaagagcagtgggactaggagctgtactccttgggttcttgggagcagcaggaagcactatgggcgcggcgtcaataacgctgacggtacaggccagacaactgttgtctggtatagtgcaacagcaaagcaatttgctgagagctatagaggcgcaacagcatatgttgcaactcacggtctggggcattaagcagctccagacaagagtcttggctatagagagatacctaaaggatcaacagctcctagggctttggggctgctctggaaaaatcatctgcaccactgctgtgccttggaactccagttggagtaataaatctcaagaagatatttgggataacatgacctggatgcagtgggatagagaaattagtaattacacaggcacaatatataggttacttgaagactcgcaaaaccagcaggagaaaaatgaaaaagatttattagcattggacagttggaaaaacttgtggaattggtttaacataacaaattggctgtggtatataaaaatattcatcatgatagtaggaggcttgataggtttgagaataatttttggtgtactcgctatagtgaaaagagttaggcagggatactcacctttgtcgtttcagacccttaccccaagcccgaggggtcccgacaggctcggaagaatcgaagaagaaggtggagagcaagacaaagacagatccattcgattagtgagcggattcttagcacttgcctgggacgatctgcggagcctgtgcctcttcagctaccaccacttgagagacttcatattgattgcagcgagagcagcggaacttctgggacgcagcagtctcaggggactgcagagagggtgggaagcccttaagtatctgggaaatcttgtgcagtatgggggtctggagctaaaaagaagtgctattaaactgtttgataccatagcaatagcagtagctgaaggaacagataggattcttgaagtaatacagagaatttgtagagctatccgccacatacctataagaataagacagggctttgaagcagctttgcaataa

SEQ ID NO: 9 - DNA, 2577 bp, Simian immunodeficiency virus
  SIV gp160
  modified_base (1)..(2577): n = g, a, c or t
atgaggaagccgatacatattatttggggtctggctttgctaatccagtttatagagaaggggacgaatgaagactatgtaacagtattctatggagtccctgtctggagaaatgcgacacctactctattttgtgccacaaatgcctccatgacaagtacagaggtgcacaatgtatgggcaactaccagttgtgtgccaatagatccagatcctattgtagttaggctcaatacctcagtctggtttaatgcttataaaaattatatggtagaaagtatgacagaagatatgntacaattattccaacaaagccataagccatgtgtaaaactaacacctatgtgtataaaaatgaattgtacaggatacaatggaacacctacaacaccaagtacaacaacaagtacagtaacaccaaagacaacaacaccaatagtagatggcatgaagctacaagaatgtaactttaatcagagcacaggatttaaagataagaaacaaaaaatgaaagccatattttataaaggagatcttatgaagtgtcaggacaacaatgagactaactgctattacttatggcactgcaacaccacaactatcacacaatcctgtgaaaagtctacttttgaaccaattcctatacattattgtgctccagcaggatatgctatattgagatgtgaagatgaggattttacaggagtagggatgtgtaaaaatgtctcagtagtacattgcactcatggaataagcccaatggtggcaacatggttactattaaatggaacttaccaaacaaacacttcagtagtaatgaatggtcgcaaaaatgaatctgtgcttgtaagatttggaaaagaattcgaaaacttaacaattacatgtataagaccaggaaataggacagtaagaaatctacaaataggaccaggaatgactttctataacgtagaaatagcaacaggagacactaggaaagcgttctgtacagtcaataagacgctatgggaacaagcacgtaacaaaacagagcacgttcttgcggagcattggaaaaaagtagacaacaaaaccaatgcgaaaacaatatggacattccaagatggagatcctgaagtaaaagtgcattggtttaattgccaaggagaattcttttattgtgatataacaccttggttcaatgccacatacacgggaaacctcatcacaaacggagccctcatagcacattgcagaattaagcagatagttaatcattggggcatagtttcaaaaggcatttacttagcccctaggagagggaatgtttcctgtacttccagcataactggaattatgttggaaggtcaaatatataatgaaactgttaaagtgtcacctgctgcaagagtagcagaccaatggagagcggagttgtccaggtaccaggtggtagagattgntcccttgtcagtagccccaacaacaggnaaaaggccagaaataaaacaacactccagacaaaaaagaggcattggaatagggctgttcttcttgggtcttctcagtgcagctggcagtacaatgggcgcagcgtcaatagcgctgacggcacagaccaggaatttgntccatggtattgtacaacagcaggccaatctgctgcaagccatagagacacagcaacatctgctacagctctcggtctggggagtaaaacaactccaggcaagaatgcttgcagtcgagaagtacctaagagatcaacaactattgagcctctggggttgtgctgacaaggtgacctgtcacactacggtgccttggaataattcctgggtaaacttcacgcaaacatgtgcaaagaacagcagtgatatacaatgtatttgggaaaatatgacatggcaagaatgggacagattagtacagaattcaacaggacagatatataatatcttacaaatagcacatgagcaacaagagagaaataaaaaggaattatatgaactagacaaatggagctcattatggaattggtttgacataacacaatggctatggtatataaaaatatttattatgatagtaggagctattgtaggactaagaattttgcttgtattagttagttgcttaagaaaggttaggcagggatatcatcctctgtcatttcagatccctacccaaaaccagcaggatccagagcagccagaagaaataagagaagaaggtggaagaaaagacaggatcaggtggagggccttgcagcacgggttcttcgcactcttgtgggtggacctgacgagcataatccagtggatctaccagatctgcagaacctgtctcttgaacctttgggcagtcctccaacacctctgcagaattactttcagactgtgcaaccatctggagaacaatctcagcaccctctggacaataatcagaactgagatcattaagaacattgacagacttgctattttggtaggggaaaaaacagatagcattcctctagctctccaaactattgtcagaatcataagggangtccctaggcgcatcagacargggttggaaattgcattwaattaa

SEQ ID NO: 10 - DNA, 262 bp, Human immunodeficiency virus
  HIV retroviral env protein
ggatcaaagtctaaagccatgtgtaaaattaaccccactctgtgttactttaagttgcgataatgtgaatattactactgccaatactaccaataccactagtaggcatgggaaactgatggagccaggagaaataaaaaactgctctttcaatatcaccacagacttgagagataagatgaagaaagaatatgcacttttttataaccttgatgtagtacaaataaatgatgataatactacctataggttgataagttgt

SEQ ID NO: 11 - DNA, 2360 bp, Ebola virus
  Reston Ebola virus glycoprotein (Gp)
atacgatgaagattaaggcgacaacgagccgaaacttcatctcttttaaagatctaacattatctgttccaaagtcatacaaggacacattcaaatcagggattgtaagctgctatttcttacctccccaaattacctatacaacatggggtcaggatatcaacttctccaattgcctcgggaacgttttcgtaaaacttcgttcttagtatgggtaatcatcctcttccagcgagcaatctccatgccgcttggtatagtgacaaatagcactctcaaagcaacagaaattgatcaattggtttgtcgggacaaactgtcatcaaccagtcagctcaagtctgtggggctgaatctggaaggaaatggaattgcaaccgatgtcccatcagcaacaaaacgctggggatttcgttcaggtgtgcctcccaaggtggtcagctatgaagccggagaatgggcagaaaattgctacaatctggagatcaaaaagtcagacggaagtgaatgcctccctctccctcccgacggtgtacgaggattccctagatgtcgctatgtccacaaagttcaaggaacaggtccttgtcccggtgacttagctttccataaaaatggggcttttttcttgtatgatagattggcctcaactgtcatctaccgagggacaacttttgctgaaggtgtcgtagcttttttaattctgtcagagcccaagaagcatttttggaaggctacaccagctcatgaaccggtgaacacaacagatgattccacaagctactacatgaccctgacactcagctacgagatgtcaaattttgggggcaatgaaagtaacaccctttttaaggtagacaaccacacatatgtgcaactagatcgtccacacactccgcagttccttgttcagctcaatgaaacacttcgaagaaataatcgccttagcaacagtacagggagattgacttggacattggatcctaaaattgaaccagatgttggtgagtgggccttctgggaaactaaaaaaacttttcccaacaacttcatggagaaaacttgcatttccaaattctatcaacccacaccaacaactcctcagatcagagcccggcgggaactgtccaaggaaaaattagctaccacccacccgccaacaactccgagctggttccaacggattcccctccagtggtttcagtgctcactgcaggacggacagaggaaatgtcgacccaaggtctaaccaacggagagacaatcacaggtttcaccgcgaacccaatgacaaccaccattgccccaagtccaaccatgacaagcgaggttgataacaatgtaccaagtgaacaaccgaacaacacagcatccattgaagactcccccccatcggcaagcaacgagacaatttaccactccgagatggatccgatccaaggctcgaacaactccgcccagagcccacagaccaagaccacgccagcacccacaacatccccgatgacccaggacccgcaagagacggccaacagcagcaaaccaggaaccagcccaggaagcgcagccggaccaagtcagcccggactcactataaatacagtaagtaaggtagctgattcactgagtcccaccaggaaacaaaagcgatcggttcgacaaaacaccgctaataaatgtaacccagatctttactattggacagctgttgatgagggggcagcagtaggattggcatggattccatatttcggacctgcagcagaaggcatctacattgagggtgtaatgcataatcagaatgggcttatttgcgggctacgtcagctagccaatgaaactacccaggctcttcaattatttctgcgggccacaacagaactgaggacttactcacttcttaacagaaaagctattgattttcttcttcaacgatggggaggtacctgtcgaatcctaggaccatcttgttgcattgagccacatgattggacaaaaaatattactgatgaaattaaccaaattaaacatgactttattgacaatcccctaccagaccacggagatgatcttaatctatggacaggttggagacaatggatcccggctggaattgggattattggagttataattgctataatagccctactttgtatatgtaagattttgtgttgatttattctgagatctgagagagaaaaatctcagggttactctaaggagaaatattatttttaaaatttacttgaatgctgaccacttatcttaaatgagcaattaataatatgtttttctgcttctttgcttgatttacaatatgatatttctcttaataatgattaatatattaagaaaaa

SEQ ID NO: 12 - DNA, 1733 bp, Influenza A virus
  Influenza A virus (A/duck/Alberta/35/76(H1N1)) haemagglutinin (HA1 and HA2 chains)
agcaaaagcaggggataatcaaatcaatcgagatggaagcaaaactatttgtactattctgtacattcactgtactgaaagctgacaccatctgtgtgggctaccatgcaaacaactctacagacactgttgacacagtactggaaaagaatgtgaccgtgactcactcagtgaatttgctcgaagacagccataatgggaaactctgcagcctgaacgggatagctcccctacaactgggaaagtgcaatgtggcgggatggctcctgggcaatccagagtgtgatcttctactcactgcaaactcatggtcctacataatagaaacttcaaactcagaaaacggaacatgctaccccggtgaattcatagattatgaagaattaagagagcagctaagttcaatttcttcatttgaaaaatttgaaattttcccgaaggcaagctcatggccaaatcatgagacaactaaaggtgttacagctgcatgctcttactctggagccagcagtttttaccggaatttgctgtggataacaaagaaagggacttcatatccaaaactcagcaaatcatacacgaacaataaggggaaagaagtgcttgtgctctggggggtgcaccaccctccaagtgtcagtgagcaacaaagtctataccagaatgctgatgcatacgtttcagttggatcgtcaaaatacaaccgaagattcgctccggaaatagcagctagacctgaagttagaggacaggcaggcagaatgaactattattggacactattagaccaaggagacactataacatttgaagccactgggaatttgatagcaccatggtatgctttcgcattgaataaggggtctgactctggaattataacatcagatgctccagttcacaattgtgacacaaggtgccaaacccctcatggggctttgaacagcagccttccttttcagaatgtacatcctatcactattggagaatgtcccaaatacgtcaagagcaccaaactaagaatggcaacaggactaagaaatgtcccatccattcagtccagaggactatttggagcaattgctggattcattgagggaggatggacaggcatgatagatggatggtacgggtatcatcatcagaatgagcaaggatcaggatatgctgctgatcagaaaagcacacagaatgcgatcgacgggatcacaagtaaggtgaattcggtaattgaaaagatgaacactcaattcactgcagtgggcaaagaattcaataatttagaaaggagaattgaaaatttgaataaaaaggtcgatgatggattcctggatgtttggacatacaatgccgaactgctcgtcctacttgaaaatgaaagaactctagactttcatgactccaatgtgagaaatttatatgagaaggtcaaatcgcaattgaggaataatgccaaagaaattgggaatggttgttttgagttctaccacaagtgtgatgatgagtgcatggaaagtgtgaagaacggcacatacgactaccccaagtattcagaagagtccaaattgaatcgagaagaaatagacggggtgaaactagaatcaatgggagtttatcaaattttggcgatctattccacagtcgccagttctctagtcttgttagtctcctggggggcaatcagcttctggatgtgctctaatgggtcattgcaatgcagaatatgcatttaa

SEQ ID NO: 13 - DNA, 20 bp, Artificial Sequence
  Description of Artificial Sequencesynthetic amplification primer for ribonucleotide reductase (RR) domain and right-flanking region of Herpes simplex virus-2 (HSV-2) ribonucleotide reductase large subunit (ICP10, RR1)
acacgccctatcatctgagg

SEQ ID NO: 14 - DNA, 20 bp, Artificial Sequence
  Description of Artificial Sequencesynthetic amplification primer for ribonucleotide reductase (RR) domain and right-flanking region of Herpes simplex virus-2 (HSV-2) ribonucleotide reductase large subunit (ICP10, RR1)
aacatgatgaaggggcttcc

SEQ ID NO: 15 - protein, 502 aa, human herpesvirus 2
  Herpes simplex virus type 2 (HSV-2) ribonucleotide reductase (ICP10, RR1)
  MOD_RES (280): Xaa = any amino acid
MANRPAASALAGARSPSERQEPREPEVAPPGGDHVFCRKVSGVMVLSSDPPGPAAYRISDSSFVQCGSNCSMIIDGDVARGHLRDLEGATSTGAFVAISNVAAGGDGRTAVVALGGTSGPSATTSVGTQTSGEFLHGNPRTPEPQGPQAVPPPPPPPFPWGHECCARRDARGGAEKDVGAAESWSDGPSSDSETEDSDSSDEDTGSGSETLSRSSSIWAAGATDDDDSDSDSRSDDSVQPDVVVRRRWSDGPAPVAFPKPRRPGDSPGNPGLGAAPGRAXPRRTRARRPTPIPAHAAAPQADVAPVLDGQPTVGTDPGYPVPLELTPENAEAVARFLGDAVDREPALMLEYFCRCAREESKRVPPRTFGSAPRLTEDDFGLLNYALAEMRRLCLDLPPVPPNAYTPYHLREYATRLVNGFKPLVRRSARLYRILGILVHLRIRTREASFEEWMRSKEVDLDFGLTERLREHEAQLMILAQALNPYDCLIHSTPNTLVERGLQ

SEQ ID NO: 16 - protein, 323 aa, Homo sapiens
  exemplary human CD47 (GenBank Accession No. Q08722.1 (GI1171879))
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 17 - DNA, 3429 bp, human herpesvirus 2
  Herpes simplex virus type 2 (HSV-2) ribonucleotide reductase (ICP10, RR1) wild-type gene
atggccaaccgccctgccgcatccgccctcgccggagcgcggtctccgtccgaacgacaggaaccccgggagcccgaggtcgccccccctggcggcgaccacgtgttttgcaggaaagtcagcggcgtgatggtgctttccagcgatccccccggccccgcggcctaccgcattagcgacagcagctttgttcaatgcggctccaactgcagtatgataatcgacggagacgtggcgcgcggtcatttgcgtgacctcgagggcgctacgtccaccggcgccttcgtcgcgatctcaaacgtcgcagccggcggggatggccgaaccgccgtcgtggcgctcggcggaacctcgggcccgtccgcgactacatccgtggggacccagacgtccggggagttcctccacgggaacccaaggacccccgaaccccaaggaccccaggctgtccccccgccccctcctcccccctttccatggggccacgagtgctgcgcccgtcgcgatgccaggggcggcgccgagaaggacgtcggggccgcggagtcatggtcagacggcccgtcgtccgactccgaaacggaggactcggactcctcggacgaggatacgggttcggagacgctgtctcgatcctcttcgatctgggccgcaggggcgactgacgacgatgacagcgactccgactcgcggtcggacgactccgtgcagcccgacgttgtcgttcgtcgcagatggagcgacggccccgcccccgtggcctttcccaagccccggcgccccggcgactcccccggaaaccccggcctgggcgccggcaccgggccgggctccgcgacggacccgcgcgcgtcggccgactccgattccgcggcccacgccgccgcaccccaggcggacgtggcgccggttctggacagccagcccactgtgggaacggaccccggctacccagtccccctagaactcacgcccgagaacgcggaggcggtggcgcggtttctgggggacgccgtcgaccgcgagcccgcgctcatgctggagtacttctgtcggtgcgcccgcgaggagagcaagcgcgtgcccccacgaaccttcggcagcgccccccgcctcacggaggacgactttgggctcctgaactacgcgctcgctgagatgcgacgcctgtgcctggaccttcccccggtcccccccaacgcatacacgccctatcatctgagggagtatgcgacgcggctggttaacgggttcaaacccctggtgcggcggtccgcccgcctgtatcgcatcctgggggttctggtccacctgcgcatccgtacccgggaggcctcctttgaggaatggatgcgctccaaggaggtggacctggacttcgggctgacggaaaggcttcgcgaacacgaggcccagctaatgatcctggcccaggccctgaacccctacgactgtctgatccacagcaccccgaacacgctcgtcgagcgggggctgcagtcggcgctgaagtacgaagagttttacctcaagcgcttcggcgggcactacatggagtccgtcttccagatgtacacccgcatcgccgggtttctggcgtgccgggcgacccgcggcatgcgccacatcgccctggggcgacaggggtcgtggtgggaaatgttcaagttctttttccaccgcctctacgaccaccagatcgtgccgtccacccccgccatgctgaacctcggaacccgcaactactacacgtccagctgctacctggtaaacccccaggccaccactaaccaggccaccctccgggccatcaccggcaacgtgagcgccatcctcgcccgcaacgggggcatcgggctgtgcatgcaggcgttcaacgacgccagccccggcaccgccagcatcatgccggccctgaaggtcctcgactccctggtggcggcgcacaacaaacagagcacgcgccccaccggggcgtgcgtgtacctggaaccctggcacagcgacgttcgggccgtgctcagaatgaagggcgtcctcgccggcgaggaggcccagcgctgcgacaacatcttcagcgccctctggatgccggacctgttcttcaagcgcctgatccgccacctcgacggcgagaaaaacgtcacctggtccctgttcgaccgggacaccagcatgtcgctcgccgactttcacggcgaggagttcgagaagctgtacgagcacctcgaggccatggggttcggcgaaacgatccccatccaggacctggcgtacgccatcgtgcgcagcgcggccaccaccggaagccccttcatcatgtttaaggacgcggtaaaccgccactacatctacgacacgcaaggggcggccatcgccggctccaacctctgcaccgagatcgtccacccggcctccaagcgatccagtggggtctgcaacctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggcattggcatgcagggcctgcacacggcgtgcctcaagatgggcctggatctggagtcggccgagttccgggacctgaacacacacatcgccgaggtgatgctgctcgcggccatgaagaccagtaacgcgctgtgcgttcgcggggcgcgtcccttcagccactttaagcgcagcatgtaccgggccggccgctttcactgggagcgcttttcgaacgccagcccgcggtacgagggcgagtgggagatgctacgccagagcatgatgaaacacggcctgcgcaacagccagttcatcgcgctcatgcccaccgccgcctcggcccagatctcggacgtcagcgagggctttgcccccctgttcaccaacctgttcagcaaggtgaccagggacggcgagacgctgcgccccaacacgctcttgctgaaggaactcgagcgcacgttcggcgggaagcggctcctggacgcgatggacgggctcgaggccaagcagtggtctgtggcccaggccctgccttgcctggaccccgcccaccccctccggcggttcaagacggccttcgactacgaccaggaactgctgatcgacctgtgtgcagaccgcgccccctatgttgatcacagccaatccatgactctgtatgtcacagagaaggcggacgggacgctccccgcctccaccctggtccgccttctcgtccacgcatataagcgcggcctgaagacggggatgtactactgcaaggttcgcaaggcgaccaacagcggggtgttcgccggcgacgacaacatcgtctgcacaagctgcgcgctgtaa

SEQ ID NO: 18 - DNA, 2961 bp, Artificial Sequence
  Description of Artificial Sequencemodified Herpes simplex virus type 2 (HSV-2) ribonucleotide reductase (ICP10, RR1) gene with some amino-terminal serine/threonine protein kinase (PK) domain deleted
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagagcggccgcttaattaaccacacgccctatcatctgagggagtatgcgacgcggctggttaacgggttcaaacccctggtgcggcggtccgcccgcctgtatcgcatcctgggggttctggtccacctgcgcatccgtacccgggaggcctcctttgaggaatggatgcgctccaaggaggtggacctggacttcgggctgacggaaaggcttcgcgaacacgaggcccagctaatgatcctggcccaggccctgaacccctacgactgtctgatccacagcaccccgaacacgctcgtcgagcgggggctgcagtcggcgctgaagtacgaagagttttacctcaagcgcttcggcgggcactacatggagtccgtcttccagatgtacacccgcatcgccgggtttctggcgtgccgggcgacccgcggcatgcgccacatcgccctggggcgacaggggtcgtggtgggaaatgttcaagttctttttccaccgcctctacgaccaccagatcgtgccgtccacccccgccatgctgaacctcggaacccgcaactactacacgtccagctgctacctggtaaacccccaggccaccactaaccaggccaccctccgggccatcaccggcaacgtgagcgccatcctcgcccgcaacgggggcatcgggctgtgcatgcaggcgttcaacgacgccagccccggcaccgccagcatcatgccggccctgaaggtcctcgactccctggtggcggcgcacaacaaacagagcacgcgccccaccggggcgtgcgtgtacctggaaccctggcacagcgacgttcgggccgtgctcagaatgaagggcgtcctcgccggcgaggaggcccagcgctgcgacaacatcttcagcgccctctggatgccggacctgttcttcaagcgcctgatccgccacctcgacggcgagaaaaacgtcacctggtccctgttcgaccgggacaccagcatgtcgctcgccgactttcacggcgaggagttcgagaagctgtacgagcacctcgaggccatggggttcggcgaaacgatccccatccaggacctggcgtacgccatcgtgcgcagcgcggccaccaccggaagccccttcatcatgtttaaggacgcggtaaaccgccactacatctacgacacgcaaggggcggccatcgccggctccaacctctgcaccgagatcgtccacccggcctccaagcgatccagtggggtctgcaacctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggcattggcatgcagggcctgcacacggcgtgcctcaagatgggcctggatctggagtcggccgagttccgggacctgaacacacacatcgccgaggtgatgctgctcgcggccatgaagaccagtaacgcgctgtgcgttcgcggggcgcgtcccttcagccactttaagcgcagcatgtaccgggccggccgctttcactgggagcgcttttcgaacgccagcccgcggtacgagggcgagtgggagatgctacgccagagcatgatgaaacacggcctgcgcaacagccagttcatcgcgctcatgcccaccgccgcctcggcccagatctcggacgtcagcgagggctttgcccccctgttcaccaacctgttcagcaaggtgaccagggacggcgagacgctgcgccccaacacgctcttgctgaaggaactcgagcgcacgttcggcgggaagcggctcctggacgcgatggacgggctcgaggccaagcagtggtctgtggcccaggccctgccttgcctggaccccgcccaccccctccggcggttcaagacggccttcgactacgaccaggaactgctgatcgacctgtgtgcagaccgcgccccctatgttgatcacagccaatccatgactctgtatgtcacagagaaggcggacgggacgctccccgcctccaccctggtccgccttctcgtccacgcatataagcgcggcctgaagacggggatgtactactgcaaggttcgcaaggcgaccaacagcggggtgttcgccggcgacgacaacatcgtctgcacaagctgcgcgctgtaa

SEQ ID NO: 19 - DNA, 2247 bp, Artificial Sequence
  Description of Artificial Sequencemodified Herpes simplex virus type 2 (HSV-2) ribonucleotide reductase (ICP10, RR1) gene with amino-terminal serine/threonine protein kinase (PK) domain deleted
atgagcggccgcttaattaaccacacgccctatcatctgagggagtatgcgacgcggctggttaacgggttcaaacccctggtgcggcggtccgcccgcctgtatcgcatcctgggggttctggtccacctgcgcatccgtacccgggaggcctcctttgaggaatggatgcgctccaaggaggtggacctggacttcgggctgacggaaaggcttcgcgaacacgaggcccagctaatgatcctggcccaggccctgaacccctacgactgtctgatccacagcaccccgaacacgctcgtcgagcgggggctgcagtcggcgctgaagtacgaagagttttacctcaagcgcttcggcgggcactacatggagtccgtcttccagatgtacacccgcatcgccgggtttctggcgtgccgggcgacccgcggcatgcgccacatcgccctggggcgacaggggtcgtggtgggaaatgttcaagttctttttccaccgcctctacgaccaccagatcgtgccgtccacccccgccatgctgaacctcggaacccgcaactactacacgtccagctgctacctggtaaacccccaggccaccactaaccaggccaccctccgggccatcaccggcaacgtgagcgccatcctcgcccgcaacgggggcatcgggctgtgcatgcaggcgttcaacgacgccagccccggcaccgccagcatcatgccggccctgaaggtcctcgactccctggtggcggcgcacaacaaacagagcacgcgccccaccggggcgtgcgtgtacctggaaccctggcacagcgacgttcgggccgtgctcagaatgaagggcgtcctcgccggcgaggaggcccagcgctgcgacaacatcttcagcgccctctggatgccggacctgttcttcaagcgcctgatccgccacctcgacggcgagaaaaacgtcacctggtccctgttcgaccgggacaccagcatgtcgctcgccgactttcacggcgaggagttcgagaagctgtacgagcacctcgaggccatggggttcggcgaaacgatccccatccaggacctggcgtacgccatcgtgcgcagcgcggccaccaccggaagccccttcatcatgtttaaggacgcggtaaaccgccactacatctacgacacgcaaggggcggccatcgccggctccaacctctgcaccgagatcgtccacccggcctccaagcgatccagtggggtctgcaacctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggcattggcatgcagggcctgcacacggcgtgcctcaagatgggcctggatctggagtcggccgagttccgggacctgaacacacacatcgccgaggtgatgctgctcgcggccatgaagaccagtaacgcgctgtgcgttcgcggggcgcgtcccttcagccactttaagcgcagcatgtaccgggccggccgctttcactgggagcgcttttcgaacgccagcccgcggtacgagggcgagtgggagatgctacgccagagcatgatgaaacacggcctgcgcaacagccagttcatcgcgctcatgcccaccgccgcctcggcccagatctcggacgtcagcgagggctttgcccccctgttcaccaacctgttcagcaaggtgaccagggacggcgagacgctgcgccccaacacgctcttgctgaaggaactcgagcgcacgttcggcgggaagcggctcctggacgcgatggacgggctcgaggccaagcagtggtctgtggcccaggccctgccttgcctggaccccgcccaccccctccggcggttcaagacggccttcgactacgaccaggaactgctgatcgacctgtgtgcagaccgcgccccctatgttgatcacagccaatccatgactctgtatgtcacagagaaggcggacgggacgctccccgcctccaccctggtccgccttctcgtccacgcatataagcgcggcctgaagacggggatgtactactgcaaggttcgcaaggcgaccaacagcggggtgttcgccggcgacgacaacatcgtctgcacaagctgcgcgctgtaa

SEQ ID NO: 20 - DNA, 655 bp, Artificial Sequence
  Description of Artificial Sequenceimmediate early cytomegalovirus (CMV) constitutive promoter regulatory sequence
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatt

SEQ ID NO: 21 - protein, 123 aa, Homo sapiens
  exemplary human CD47 excluding signal sequence
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNE